(19) United States
(12) Patent Application Publication
(10) Pub. No.: US 2018/0075763 A1
(43) Pub. Date: Mar. 15, 2018

(54) SYSTEM AND METHOD OF GENERATING RECOMMENDATIONS TO ALLEVIATE LONELINESS

(71) Applicant: S. Lynne Wainfan, Long Beach, CA (US)

(72) Inventor: S. Lynne Wainfan, Long Beach, CA (US)

(21) Appl. No.: 15/705,227

(22) Filed: Sep. 14, 2017

Related U.S. Application Data
(60) Provisional application No. 62/394,802, filed on Sep. 15, 2016.

(51) Int. Cl. G09B 5/02; A61B 5/16; A61B 5/00; G09B 19/00; G09B 5/06

(52) U.S. Cl. CPC G09B 5/02; A61B 5/165; A61B 5/163; H04L 51/02; G09B 19/00; G09B 5/065; A61B 5/4803

(57) ABSTRACT

The embodiments disclose a system and method of generating personalized recommendations to alleviate loneliness including detecting, collecting, and organizing user information related to loneliness including sensing user information associated with a users' loneliness in a way that the user may remain anonymous, using user information to compute a user loneliness profile, using the user loneliness profile to compute one or more loneliness alleviation recommendations, presenting the one or more loneliness alleviation recommendations to the user, analyzing effectiveness of the one or more loneliness alleviation recommendations, using effectiveness analysis results and a user's loneliness profile to compute and store updates to databases and algorithms that compute all users' loneliness alleviation recommendations, converting a user's updated loneliness profile from updated user information and one or more loneliness alleviation recommendations effectiveness analysis results and using the updated user information and updated user loneliness profile to compute one or more updated loneliness alleviation recommendations.

### CROSS-REFERENCE TO RELATED APPLICATIONS

This application is based on U.S. Provisional Patent Application Ser. No. 62/394,802 filed Sep. 15, 2016, entitled “System and Method of Generating Recommendations to Alleviate Loneliness”, by S. Lynne Wainfan.

### BACKGROUND

Loneliness is exceptionally common. Research has found that everyone is lonely at some point in their lives, but at any given time, 30% of Americans are experiencing frequent or intense loneliness. Loneliness has dire health consequences; compromised immune system; increased risk for heart and vascular disease, cancer, neurodegenerative disease and viral infections; increased blood pressure and inflammation. Beyond the significant effects on individuals' physical and mental health, loneliness has effects on society, in the form of lost productivity and increased use of the healthcare system. There are several challenges to treating loneliness, including a lack of clinical guidance and people's reluctance to seek help for loneliness. Because loneliness is so common and getting worse, with dire consequences on individuals and society, and currently difficult to treat, there is a great need for a more effective system and method to alleviate loneliness.

### BRIEF DESCRIPTION OF THE DRAWINGS

FIG. 1 shows for illustrative purposes only an overview of a system and method of generating recommendations to alleviate loneliness of one embodiment.

FIG. 2 shows a block diagram of an overview flow chart of a system and method of generating recommendations to alleviate loneliness of one embodiment.

FIG. 3 shows a block diagram of an overview flow chart of user internet/intranet connection of one embodiment.

FIG. 4 shows a block diagram of an overview an example of loneliness profile types of one embodiment.

FIG. 5 shows a block diagram of an overview an example of loneliness alleviation recommendation types of one embodiment.

FIG. 6 shows a block diagram of an overview an example of loneliness alleviation recommender algorithm of one embodiment.

FIG. 7A shows a block diagram of an overview an example of interactive network resources of one embodiment.

FIG. 7B shows a block diagram of an overview an example of user recommendations taken/not and analyzed to evaluate effectiveness of one embodiment.

FIG. 7C shows a multi-level block diagram of an overview example of top level recommendation—solve the problem of one embodiment.

FIG. 8 shows for illustrative purposes only an example of network interconnections of one embodiment.

FIG. 9 shows a block diagram of an overview flow chart of learning loop of one embodiment.

FIG. 10A shows a block diagram of an overview flow chart of collect 1st round user information of one embodiment.

FIG. 10B shows a block diagram of an overview flow chart of op-level loneliness alleviation recommendations of one embodiment.

FIG. 11A shows a block diagram of an overview flow chart of data analysis method of one embodiment.

FIG. 11B shows a block diagram of an overview flow chart of learning of one embodiment.

FIG. 12 shows for illustrative purposes only an example of Virtual Buddy and user interaction of one embodiment.

FIG. 13 shows for illustrative purposes only an example of web resource social skills training of one embodiment.

FIG. 14A shows a block diagram of an overview flow chart of loneliness alleviation recommendations application of one embodiment.

FIG. 14B shows a block diagram of an overview flow chart of convert the active user information to a loneliness profile of one embodiment.

FIG. 14C shows a block diagram of an overview flow chart of computed effectiveness results of one embodiment.

FIG. 15 shows a block diagram of an overview an example of algorithm functions to calculate loneliness alleviation recommendations of one embodiment.

FIG. 16 shows a block diagram of an overview an example of other implicit user information devices of one embodiment.

FIG. 17A shows a block diagram of an overview flow chart of a modified demographic recommender system data analysis method of one embodiment.

FIG. 17B shows a block diagram of an overview flow chart of a user professions expected loneliness groups categorization analysis process of one embodiment,

FIG. 17C shows a block diagram of an overview flow chart of a user information to loneliness profile conversion process of one embodiment.

FIG. 17D shows a block diagram of an overview flow chart of a loneliness profile vectors measure of similarity analysis process of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the embodiments.

### General Overview:

It should be noted that the descriptions that follow, for example, in terms of a system and method of generating recommendations to alleviate loneliness is described for illustrative purposes and the underlying system can apply to any number and multiple types a system and method of generating recommendations to alleviate loneliness. In one embodiment of the present invention, the user device can be configured using desktop computing devices, laptop computing devices, tablet computing devices and cellular phones. The loneliness alleviation network can be configured to include at least one digital computer with servers, digital storage devices, digital processors, algorithms, databases, communication devices, text to speech devices, image animation devices, translation devices and can be configured to include other implicit user information devices using the embodiments.

Loneliness is often defined as the difference between the social relations desired by an individual, and what he or she perceives that they have. Thus it is perception-based: subjective and personal. Loneliness has been called a public health crisis. Compared with the non-lonely, lonely people have a 45% higher risk of early death and a lifespan shortened by 14 years. Research has shown that In addition to physical health consequences, loneliness is closely related to mental illness, specifically depression and anxiety. Compared to the non-lonely, lonely people have a 64% higher risk of dementia. Lonely people visit general practitioners three times more frequently and have 30% more emergency hospital admission rates compared with the non-lonely people. There is also a contagion effect: non-lonely people get lonelier if they are around lonely people.

Loneliness worldwide is getting worse with time. One study found that in the past twenty years, the number of people who had “no one to talk to” had doubled. Loneliness is difficult to alleviate for a number of reasons, First, mental health practitioners are strongly encouraged to use the Diagnostic and Statistical Manual (DSM) classifications in their practice for four purposes: the DSM classifications are used to inform practitioners of relevant alleviations; the DSM classifications are used to provide clinical information to researchers and practitioners; the classifications are the basis for statistics that are collected for public health record keeping; and DSM provides classifications that are generally required for insurance reimbursement. Since loneliness does not have a classification in the current DSM, it is rarely treated clinically.

People are more likely to admit that they are depressed than talk about their loneliness. In addition to reluctance to talk about their loneliness, people may be unwilling to divulge information about their behavior related to loneliness, i.e. staying home all day, ending their online relationships, etc. Some are unable to divulge loneliness-related information because their behaviors may not be detected to be relevant. Increased Internet usage, alienating chat room language, a change in e-mail frequency may not be noticed or thought relevant by the user. For these reasons, many unspoken factors cannot be addressed by current alleviation modalities.

Yet another challenge with current methodology comes from the fact that research results showing ineffective alleviations are not typically published. Information about ineffective alleviations may be very useful in devising recommendations, yet that information is generally not available with the current methods. Another reason that loneliness is difficult to alleviate clinically is that it takes years, sometimes decades, for researchers to progress from initially conceiving an experiment to testing an alleviation's effectiveness to publication of results. Taking the further step of adopting alleviation methods in clinical practice is difficult for the reasons addressed earlier, and adds more years to the process.

The major reason that loneliness is currently difficult to alleviate is because it is exceptionally challenging to develop recommendations to alleviate it. This is because there are different types of loneliness, i.e. chronic vs. transient; different types of people, i.e. disabled seniors vs. friendless adolescents; different situations, i.e. isolated disabled people vs. unhappy socialites; and different types of alleviation; i.e. learning to use social media, social skills training, etc. Thus, developing an alleviation recommendation is a multi-dimensional problem which is difficult for current practitioners to implement.

Individuals often self-treat loneliness with a number of maladaptive behaviors: To distract themselves from the adverse feelings of loneliness, they may engage “too much” in activities such as drinking, taking drugs, working, having excessive sex, and partying. To protect themselves from what they perceive to be worse loneliness, they sometimes isolate themselves away from people, reject others before the other person has a chance to reject them, or stay in relationships that they would otherwise end in order not to worsen their loneliness. Conversely, loneliness can threaten existing relationships, and prevent repair. Ironically, lonely people tend to stop trying to initiate new social relations. Thus, people trying to address their own loneliness by themselves often worsen it.

Traditional psychology research uses experiments to see if one type of alleviation is effective for one type of loneliness for one type of person in one type of situation. Given the large numbers of combinations, and the length of time it takes to test each single-factor thread of the problem space, it will be a long time indeed before the numerous combinations of factors affecting numerous types of alleviations can be determined using the current methodology. In addition, this multi-factored problem requires complex mathematics to compute the best alleviation recommendations. Current treatment modalities typically use the DSM manual to recommend relatively more straightforward ways of development alleviations.

Furthermore, it would be beneficial to develop an anonymous alleviation method for people hesitant to talk about their loneliness. It would also be beneficial to collect more information on users' characteristics and behavior related to loneliness so that recommendations can be personalized for specific types of individuals with specific types of loneliness. There is also a need to develop alleviation recommendations more quickly and effectively than current practice allows. Finally, as more is learned on this multi-factored problem of loneliness, there is a need for the recommendation process to learn and adapt to new findings.

### SUMMARY OF THE INVENTION

The present invention involves systems and methods for computing and presenting a user one or more recommendations to alleviate the user's loneliness. The recommendations are personalized, based on the user's loneliness profiles. The user's loneliness profile is computed from user information from online and offline activities. Loneliness profiles include information about the type of loneliness, the user's personal characteristics and situation that pertains to loneliness. The recommendations to alleviate loneliness are computed using these loneliness profiles, a database, and an algorithm that incorporates a modified demographic recommender system or alternatively multi-dimensional structural equations and data mining techniques. The database includes loneliness alleviation information from sources such as research, clinical data, and effectiveness information from the present invention. This effectiveness information includes, but is not limited to, user acceptance of loneliness alleviation recommendations; user activity associated with the recommendations; and updated change in loneliness profiles. From the effectiveness information, the algorithms and databases associated with the user loneliness profile computation and recommendation computation are updated, both for the user and for all other users. These algorithms and databases can be updated while preserving the recommender system or structural equations that are the basis for the computation, or by changing the recommender system or structural equations. In this way, the system can effectively “learn” from previous experience,

It is an object of the present invention to generate recommendations to alleviate loneliness. The method includes the steps of: detecting, collecting, and organizing user information related to loneliness; using this information to compute user loneliness profiles; using those loneliness profiles to compute one or more loneliness alleviation recommendations; presenting those recommendations to the user; monitoring acceptance or rejection of those recommendations, computing a user's updated loneliness profiles from updated user information; using the updated user information and loneliness profiles to determine the effectiveness of the recommendations; and using that effectiveness information and loneliness profile to compute and deliver updates to databases and algorithms that compute user loneliness profiles and loneliness alleviation recommendations.

It is a further object of the present invention to provide a software method to compute customized recommendations to users to alleviate their loneliness. These recommendations fall into four types: crisis actions; solve the problem; talk to someone; or distract. Examples of crisis actions include recommending the user contact a therapist, or the application directly calling 911. Examples of solve the problem include improving the stock or flow of people whom the user comes in contact with; improve the process of establishing a relationship; or adjust perspectives to accept the situation as temporary. Examples of talk to someone include talking to a family member or friend, a professional, or a virtual buddy. A Virtual Buddy is a computer generated character initialization that can be 2D or 3D configured and has facial, skin tones, accented voice, and age appropriate characteristics that closely match the user information and any stated user preferences in a desired friend characteristics. Examples of distract include playing a game, or taking up a hobby. The computation of alleviation recommendations utilizes user loneliness profiles, which are derived from user information gathered from his or her behavior, situation, and personal characteristics. This user information can come from online or offline sources. For example: phone GPS location data that indicates a user spending more time in their home; a change in social media relationship status from married to divorced; or a change in address to a nursing home; or a user typing into the system, “I'm very lonely” would be user information related to loneliness.

It is a further object of the present invention to present recommendations to the user for ways to alleviate their loneliness.

It is a further object of the present invention to measure effectiveness of the recommendations. Effectiveness measures use information such as, but not limited to: user acceptance of recommendation; user information related to loneliness; and users' loneliness profile after the recommendation is acted upon or not.

It is an object of the present invention to use these effectiveness measures along with user information to update the database and algorithm that computes loneliness alleviation recommendations. In this way the system “learns” quickly compared with the current practice.

It is an object of the present invention to use these effectiveness measures, new research findings, and user information to update the database and algorithm that computes loneliness alleviation recommendations. In this way the system “learns” quickly compared with the current practice.

It is an object of the present invention to use these effectiveness measures, new research findings, and user information to update the database and algorithms that compute loneliness profiles from user information. This may include the insertion of new user information to collect, a result of new information on what information will be relevant or worthy of study. Similarly, the algorithm that computes loneliness profiles from user information will be updated as new information becomes available from outside research or from use of the present invention. For instance, if it is found that a user sending certain messages to loved ones is a strong predictor of loneliness, then this information will be included in the database and algorithm. In this way the system “learns” quickly compared with the current practice.

According to the present invention, a novel method of sensing loneliness profiles and recommending ways to alleviate loneliness is disclosed. [0025] According to the present invention, a novel system and method to generate recommendations to alleviate loneliness is disclosed, The method includes the steps of: detecting, collecting, and organizing user information related to loneliness; using this information to compute user loneliness profiles; using those loneliness profiles to compute one or more loneliness alleviation recommendations presenting those recommendations to the user; detecting acceptance or rejection of those recommendations, computing a user's updated loneliness profiles from updated user information; using the updated user information and loneliness profiles to determine the effectiveness of the recommendations; and using that effectiveness information and user information to compute and deliver updates to databases and algorithms that compute user loneliness profiles and compute loneliness alleviation recommendations

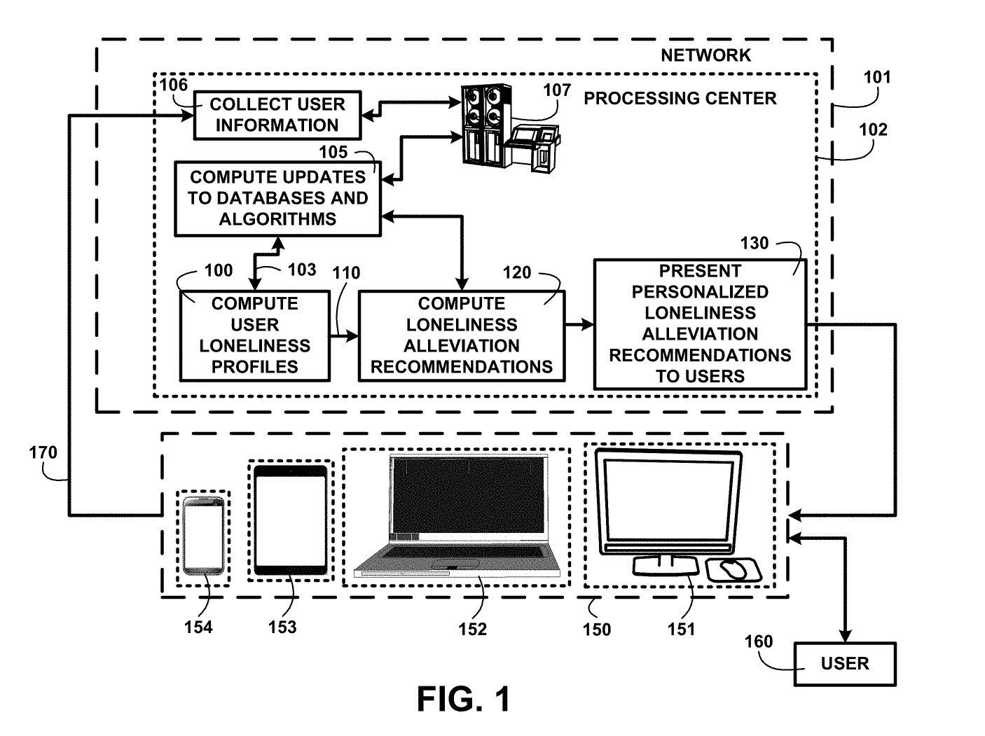

FIG. 1 shows for illustrative purposes only an overview of a system and method of generating recommendations to alleviate loneliness of one embodiment. FIG. 1 shows a network 101 with a processing center 102. The processing center 102 is used to compute updates to databases and algorithms 105 and collect user information 106 from a user device 150. The network 101 includes at least one computer with servers 107 and algorithm and databases 103. The network 101 is used to compute user loneliness profiles 100 and to compute loneliness alleviation recommendations 120 and present personalized loneliness alleviation recommendations to users 130. The personalized loneliness alleviation recommendations are presented to a user 160 through a user device 150 including desktop computing devices 151, laptop computing devices 152, tablet computing devices 153, and cellular phones 154 as well as other devices that may indicate the user's activities or characteristics or activities associated with loneliness. A user device 150 can also provide user information 170 that includes location information from a GPS signal, internet usage, and phone location. User information related to loneliness is entered by the users and transmitted to the network 101 of one embodiment.

User information 170 is organized into three categories: user's activities; personal characteristics related to loneliness; and situational information related to loneliness. User's activity information includes online actions such as browsing the internet; using e-mail, chat, or social media; playing online games; and offline activity information such as location, and phone and text message history. Personal characteristics information includes a user's contact list, mobility characteristics, gender, and age. Situational information includes relationship status, city, and employment status of one embodiment.

User information 170 is transmitted from the user device to the network 101. User information 170 is collected in two ways: passively and actively entered information. Passively-entered information includes device location from a GPS system and location history. Actively-entered information is of two sub-groupings: directly entered into the present system; or indirectly entered. Active, directly-entered information includes information such as responses to the system's queries such as, “did your wife pass away recently?” or information the user enters without being queried, such as, “I'm feeling very lonely,” Active, indirectly-entered information includes information gathered from the user's online activities such as usage of: e-mail, social media, and shopping, video, audio, browsing choices. For example, if the user's relationship status reported online changed from “married” to single, or if the user were browsing for ways to harm himself, the present system will collect that information since it relates to the user's loneliness. Similarly, the user's chat/message/email history will be collected to be analyzed for changes that indicate loneliness, Another active, indirectly-entered information example comes from offline activities, such as phone call history; and text message frequency. recipient, and content. User information includes recent information and past history. These examples illustrate the type of user information that will be collected but are not intended to be all inclusive of one embodiment.

The collected information is organized, analyzed, and computed in step 100 into a mathematical matrix called loneliness profiles, item 110. Loneliness profiles are organized into four types including crisis profile, situational profile, personal profile, and cultural profile of one embodiment.

A process to compute loneliness alleviation recommendations 120 uses the loneliness profiles as input to an algorithm and database that are used to compute loneliness alleviation recommendations 120. The algorithm has as its foundation a modified Demographic recommender system that includes an algorithm and coefficient database. Alternatively the algorithm can consist of matrix-based structural equations where the equation dimensionality is derived from data mining methodology, a coefficient database, and a weighting function. These loneliness alleviation recommendations 240 include, but are not limited to, specific loneliness alleviation recommendations within the four categories of: crisis actions; solve the problem; talk to someone; or distract. Examples of crisis actions include recommending the user contact a therapist, or the application directly calling 911. Examples of solve the problem include improving the stock or flow of people who the user comes in contact with; improve the process of establishing a relationship; or adjust perspectives to accept the situation as temporary. Examples of talk to someone include talking to a family member or friend, a professional, or a virtual buddy. Examples of distract include playing a game, or taking up a hobby. Thus, some loneliness alleviation recommendations 240 can involve offline activities and some can be online activities. The loneliness alleviation recommendation computation will also take into account information for loneliness alleviation recommendations that have not been effective previously of one embodiment.

The system can present personalized loneliness alleviation recommendations to users 130, wherein the loneliness alleviation recommendation is arrived at through computations using user information 170 from loneliness profiles; alerting the user if that severity is above a multi-factor threshold; inquiring whether the user wishes to address that loneliness; if the user elects not to address their loneliness, the system continues to gather user information, and compute the loneliness profiles. If the user elects to address their loneliness, the system displays the best loneliness alleviation recommendation to alleviate the user's loneliness. The system then inquires whether the user wants to follow up on that loneliness alleviation recommendation. If not, the system generates additional loneliness alleviation recommendations until the user either accepts a loneliness alleviation recommendation or expresses a desire to end the loneliness alleviation recommendations 240. Information on user rejection or acceptance of the recommendations is then included in the loneliness profile 110.

Algorithms compute updates to databases and algorithms from the learning that the system does about the most effective recommendations. The algorithm uses as its input the recommendations given to a user; loneliness profile 110 information pertaining to whether the user took that recommendation; and the change in a user's loneliness profile associated with the recommendations of one embodiment.

Algorithms use the above information to compute a variable called recommendation effectiveness. A simple example of the computation flow is as follows Algorithm receives information that the user accepted the recommendation. It also receives information about the user's activity related to that recommendation. Finally, the algorithm receives new or updated loneliness profiles and previous loneliness profiles. If the loneliness profiles have diminished, then the recommendation is considered to be “effective” on a sliding scale. This information is then used to update the recommender matrix, the structural equations, other algorithms, and databases for the algorithm being used. These updates are based on individual user effectiveness; effectiveness of multiple users; and new information that becomes available from outside the present system of one embodiment.

From the present system's usage, from clinical practice, or from outside research, it may turn out that the type of user information collected can be improved. For example, should a new type of online behavior predict extreme loneliness, then this new type of online behavior will be added to the list of user information 170 to collect. Similarly, weighting functions or combinatorial algorithms that are used to compute loneliness profiles may be updated as more information becomes available. These changes to the databases and algorithms calculate a updated user loneliness profile using the updated information of one embodiment.

### DETAILED DESCRIPTION

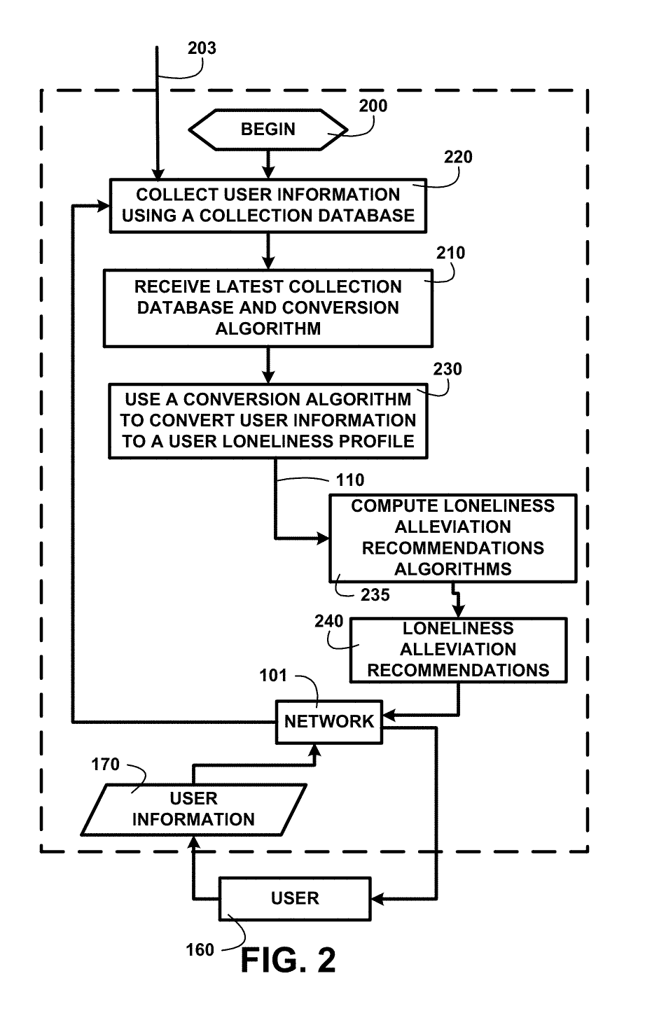

FIG. 2 shows a block diagram of an overview flow chart of a system and method of generating recommendations to alleviate loneliness of one embodiment. FIG. 2 shows algorithms and databases information 203 being used from the processing center 102 of FIG. 1 to begin 200 the computations. User information 170 from the network 101 is receive by the latest collection database and computation algorithm 210. The system begins to collect user information using a collection database 220 and use a computation algorithm to convert user information to a user loneliness profile 230. The loneliness profiles 110 are transmitted to compute loneliness alleviation recommendations algorithms 235 to produce loneliness alleviation recommendations 240. The network 101 receives and transmits user information 170 from the user 160 of one embodiment.

Updates may come from two areas of learning: better user information items to collect; and better ways to convert user information to loneliness profiles. Also, the recommender system algorithm and database can be updated. Using the latest database and algorithm, the process directs the user information 170 collection databases on what to collect, and it collects user information 170. The process uses the latest computation algorithm to convert user information 170 to loneliness profiles 110. These loneliness profiles are then used to compute loneliness alleviation recommendations 240 of one embodiment.

### User Internet/Intranet Connection:

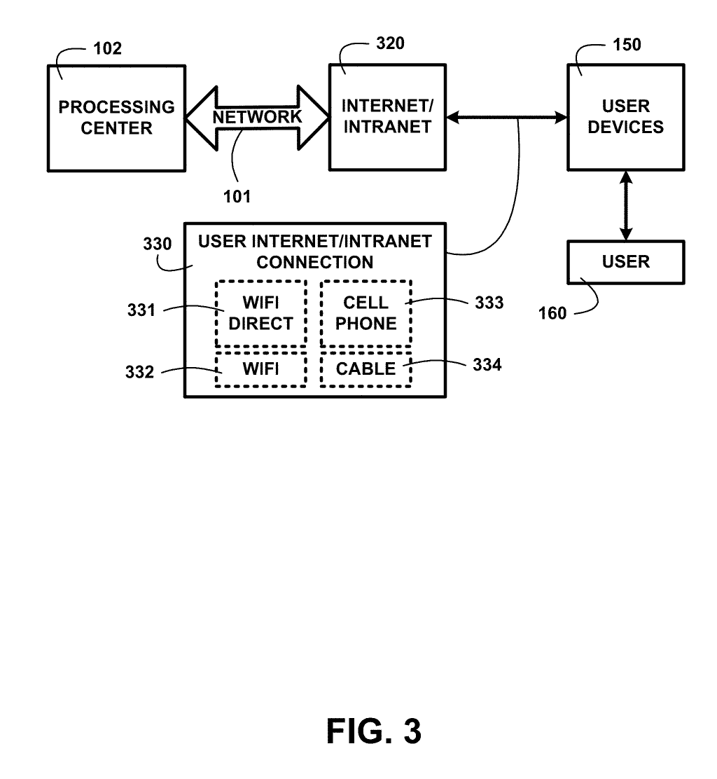

FIG. 3 shows a block diagram of an overview flow chart of user internet/intranet connection of one embodiment. FIG. 3 shows the processing center 102 and network 101 connected to internet/intranet 320 systems. The internet/intranet 320 systems provide the user internet/intranet connection 330 including WIFI direct 331, WIFI 332, cell phone 333, and cable 334 to user devices 150 to communicate with the user 160.

The processing center 102 provides authorization for the user to access the system. It also downloads software to user devices, both initially and as updates are desired. The processing center 102 also ensures anonymity for the user, protecting his or her identity and information from being associated and revealed. Finally, the processing center also queries and sets user requirements and limitations for the type of information to be gathered, for example, the user may elect to not have the system detect user loneliness information from e-mails, or contact list information from his or her phone. The user 160 using the user devices 150 controls the information accessed via the network 101. In another embodiment the user may be an agent of the person wishing to receive loneliness alleviation recommendations. This is useful, for instance, if the person does not use digital devices. This agent might be a friend, family member, social worker, healthcare professional, volunteer, or anyone else who could provide information about he person wishing to receive the recommendations of one embodiment.

In yet another embodiment information, algorithms, and databases of the present invention to be stored on one or more user devices 150. In this embodiment, the processing center 102 connects with the user devices 150 through a relatively less frequent type of connection in order to perform the functions described for collecting user information and recommendation effectiveness information which are stored on the user's device, and uploaded to the processing center as a connection becomes available of one embodiment.

Thus, the major challenges of current treatments for loneliness can be addressed within the system and method of generating recommendations to alleviate loneliness that detects user information related to loneliness and uses mathematical computations to derive the best recommendations for that user to alleviate his or her loneliness. The system and method of generating recommendations to alleviate loneliness allows more factors to be considered than are considered practical in typical clinical manuals; it allows sensitive information, which a patient may not want to reveal, to be collected and used anonymously; it produces recommendations that are based on a larger number of user loneliness profiles than could be done by hand; it updates the algorithms and databases of those recommendations very quickly compared to the current method of research, publication, and transferal to clinical practice; and it offers loneliness alleviation recommendations for a fraction of the cost of current clinical treatment. In addition, when the system and method of generating recommendations to alleviate loneliness is used to help users alleviate their loneliness, the physical and mental healthcare issues associated with loneliness will decline, reducing the associated cost to society of one embodiment.

### Loneliness Profile Types:

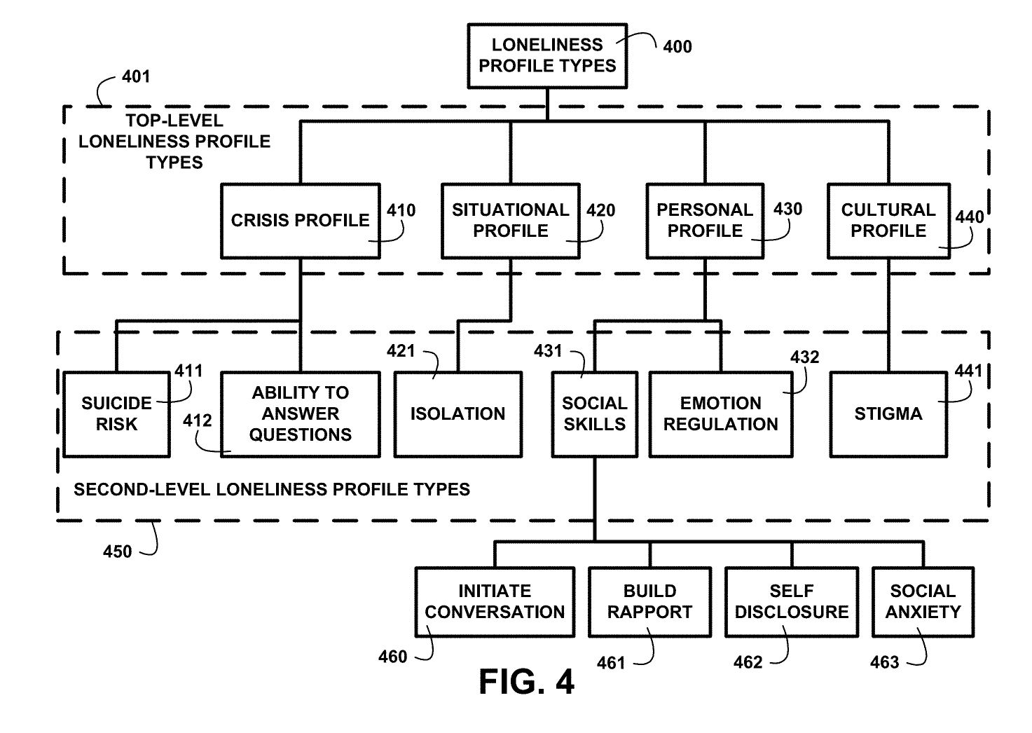

FIG. 4 shows a block diagram of an overview an example of loneliness profile types of one embodiment. FIG. 4 shows loneliness profile types 400 including top-level loneliness profile types 401. Top-level loneliness profile types 401 include crisis profile 410, situational profile 420, personal profile 430, and cultural profile 440. The loneliness profile types 400 also includes second-level loneliness profile types 450 for example related to crisis profile 410 are suicide risk 411 and ability to answer questions 412. Related to situational profile 420 is isolation 421. The top-level loneliness profile personal profile 430 has related second-level loneliness profile types 450 including emotion regulation 432 and social skills 431. Social skills 431 include initiate conversation 460, build rapport 461, self disclosure 462, and social anxiety 463. Top-level loneliness profile cultural profile 440 has stigma 441 related of one embodiment.

### Loneliness Alleviation Recommendation Types:

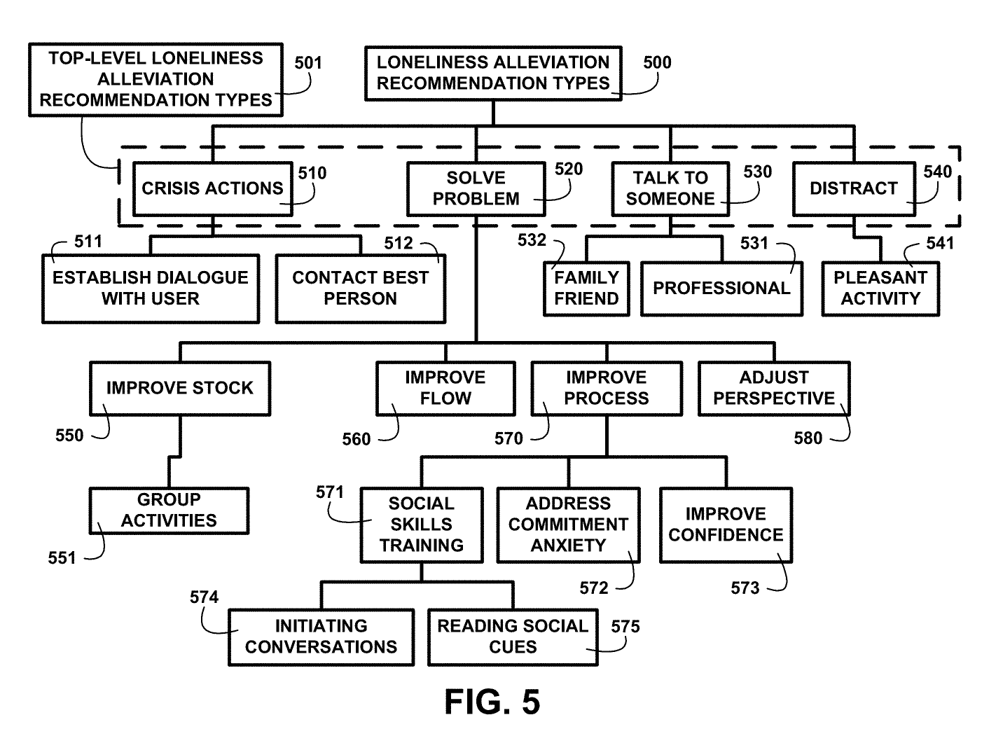

FIG. 5 shows a block diagram of an overview an example of loneliness alleviation recommendation types of one embodiment. FIG, 5 shows loneliness alleviation recommendation types 500. The loneliness alleviation recommendation types 500 include top-level loneliness alleviation recommendation types 501 including crisis actions 510, solve problem 520, talk to someone 530, and distract 540. Crisis actions 510 include establish dialogue with user 511 and contact best person 512. Top-level loneliness alleviation recommendation type solve problem 520, improve stock 550, group activities 551, improve flow 560, improve process 570, social skills training 571, initiating conversations 574, reading social cues 575, address commitment anxiety 572, improve confidence 573, and adjust perspective 580. Top-level loneliness alleviation recommendation type talk to someone 530, includes family friend 532 and professional 531. Distract 540 includes pleasant activity 541 of one embodiment.

### Loneliness Alleviation Recommender Algorithm:

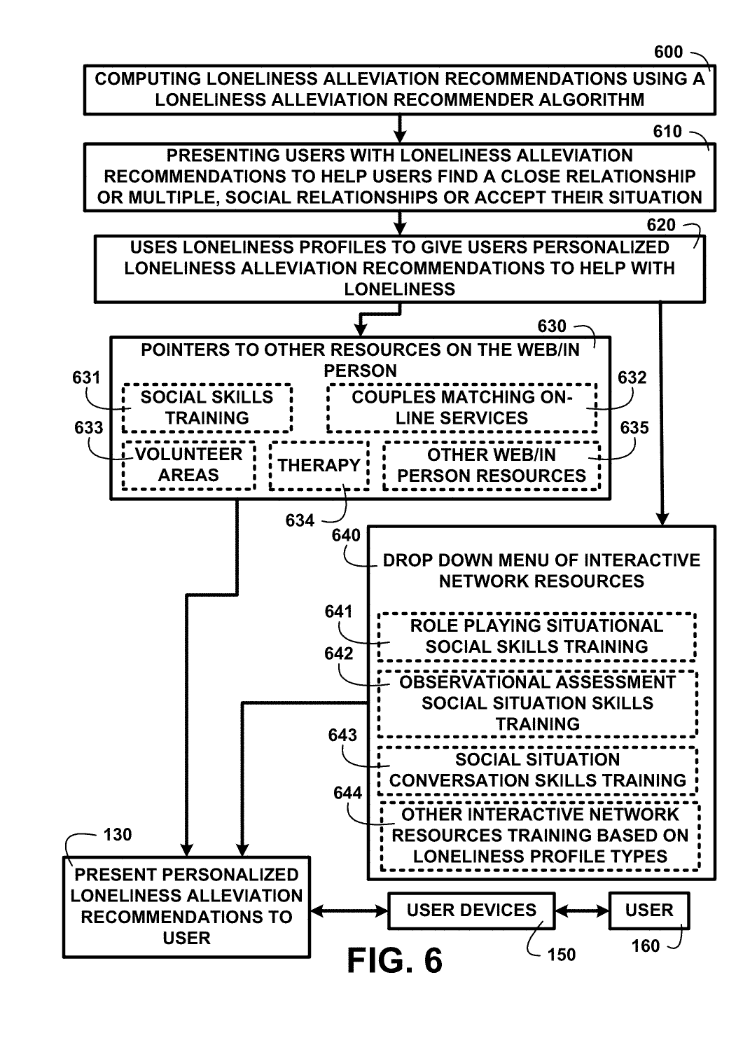

FIG. 6 shows a block diagram of an overview an example of loneliness alleviation recommender algorithm of one embodiment. FIG. 6 shows a continuation from FIG. 5 including a process to compute loneliness alleviation recommendations using a loneliness alleviation recommender algorithm 600. Presenting users with loneliness alleviation recommendations to help users find a close relationship or multiple social relationships or accept their situation 610. The system and method of generating recommendations to alleviate loneliness uses loneliness profiles that are used to compute and present users personalized loneliness alleviation recommendations to help with loneliness 620. The loneliness alleviation recommendations provides pointers to other resources on the web/in person 630 including social skills training 631, couples matching on-line services 632, volunteer areas 633, therapy 634 and other web/in person resources 635. The loneliness alleviation recommendations can also point to resources within the app, for instance, personalized, interactive social skills training that is not available elsewhere. The loneliness alleviation recommendations provides a drop-down menu of interactive network resources 640 that include on site role playing situational social skills training 641, observational assessment social situation skills training 642, social situation conversation skills training 643, and other interactive network resources training based on loneliness profile types 644. The system and method of generating recommendations to alleviate loneliness includes a process to present personalized loneliness alleviation recommendations to users 130 through user devices 150 to the user 160 of one embodiment.

### Interactive Network Resources:

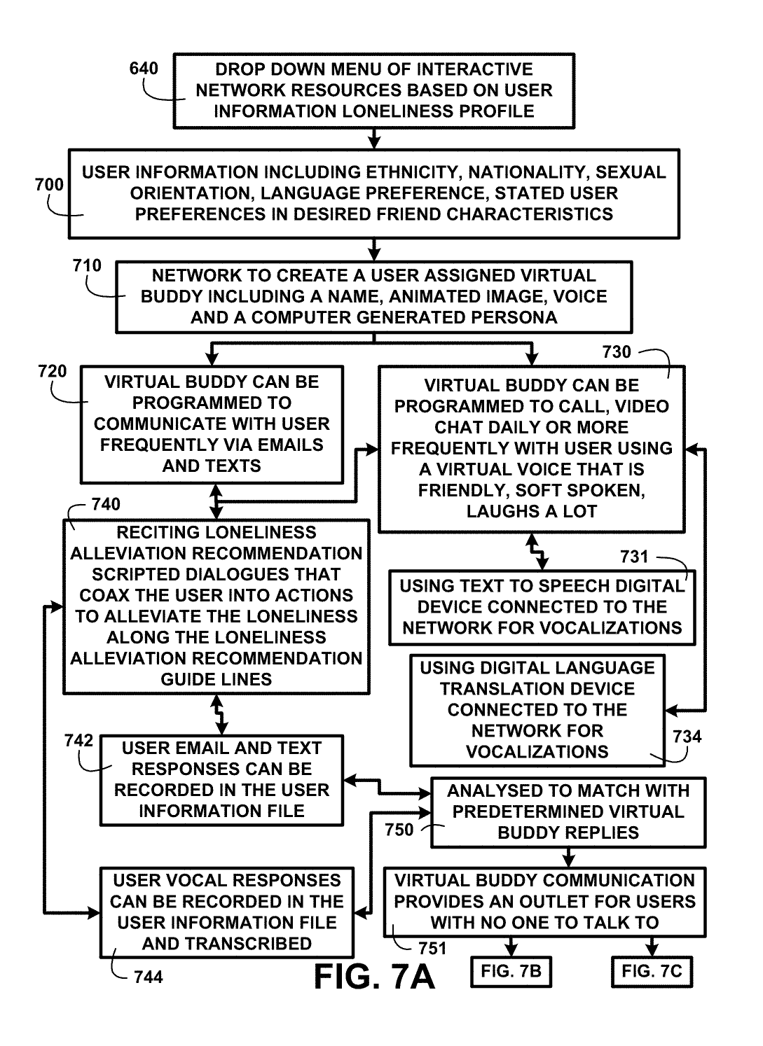

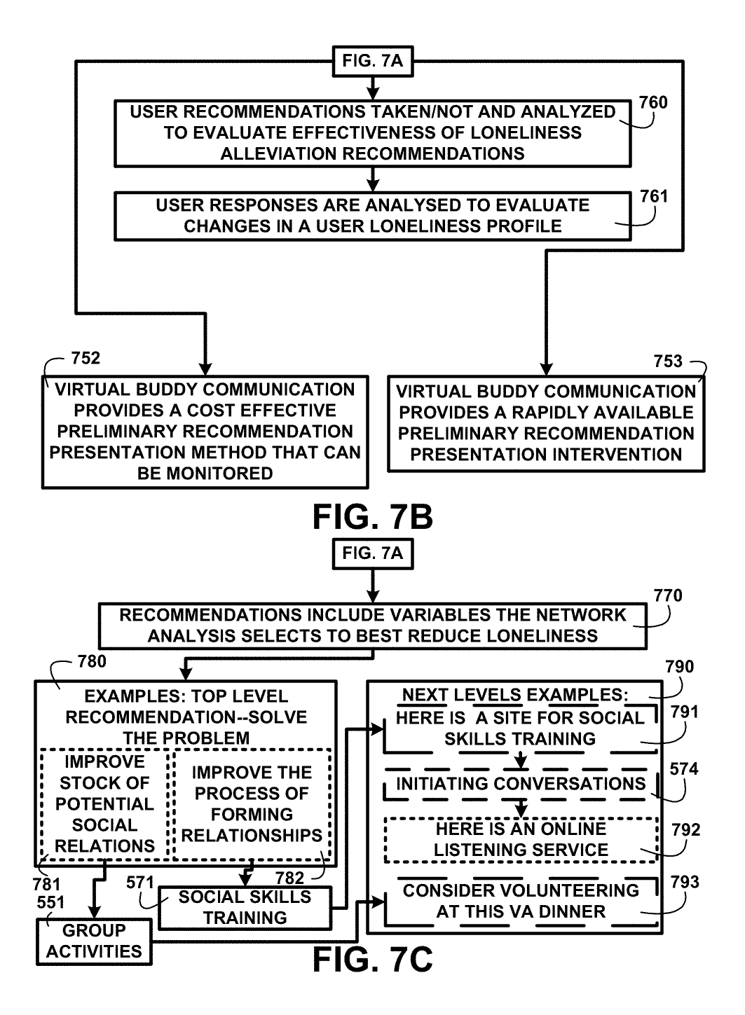

FIG. 7A shows a block diagram of an overview an example of interactive network resources of one embodiment, FIG. 7A shows the drop down menu of interactive network resources based on user information loneliness profile 640. User information including ethnicity, nationality, sexual orientation, language preference, stated user preferences in desired friend characteristics 700 is used by the network to create a user assigned Virtual Buddy including a name, animated image, voice and a computer generated persona 710. A Virtual Buddy is a computer generated character initialization that can be 2D or 3D configured and has facial, skin tones, accented voice, and age appropriate characteristics that closely match the user information and any stated user preferences in a desired friend characteristics. A Virtual Buddy can be programmed to communicate with user frequently via emails and texts 720, A Virtual Buddy can be programmed to call, video chat daily or more frequently with user using a virtual voice that is friendly, soft spoken, laughs a lot 730. The network can use text to speech digital devices connected to the network for vocalizations 731. The network can use digital language translation devices connected to the network for Virtual Buddy vocalizations 734 in a user preferred language. The Virtual Buddy can be programmed for reciting loneliness alleviation recommendation scripted dialogues that coax the user into actions to alleviate the loneliness along the loneliness alleviation recommendation guide lines 740. Virtual Buddy communications provide an outlet for users with no one to talk to 751 and can provide a distraction also for example by showing a funny video, playing a game, reading a user's favorite book or poem and other distractions for the user. Virtual Buddy interactions with a user can provide directly some of the recommendations under “distract” or “talk with someone. User email and text responses can be recorded in the user information file 742. User vocal responses can be recorded in the user information file and transcribed 744. User responses are analyzed to match with predetermined Virtual Buddy replies 750 of one embodiment. The description continues on FIG. 7B and FIG. 7C.

### User Responses Analyzed to Evaluate Effectiveness:

FIG. 7B shows a block diagram of an overview an example of user recommendations taken/not and analyzed to evaluate effectiveness of one embodiment. FIG. 7B shows a continuation from FIG. 7A where user responses for example accepting a loneliness alleviation recommendation to talk to someone and the user talking to an online listening service are analyzed to evaluate effectiveness of loneliness alleviation recommendations 760 are recorded in the user information file. User recommendations taken/not are analyzed to evaluate changes in a user loneliness profile 761. User Information would be monitored, loneliness profile computed, and the changes in loneliness profile would be the effectiveness for that initial Loneliness Profile for that Recommendation. Virtual Buddy communication provides a cost effective preliminary recommendation presentation method that can be monitored 752. Virtual Buddy communication provides a rapidly available preliminary recommendation presentation intervention 753 of one embodiment.

### Examples: Top Level—Solve the Problem:

FIG. 7C shows a multi-level block diagram of an overview example of top level recommendation—solve the problem of one embodiment. FIG. 7C shows a continuation from FIG. 7A showing recommendations the algorithms compute including the loneliness alleviation recommender algorithm to best reduce loneliness 770. Examples: top level recommendation—solve the problem 780 include two lower-level recommendations: improve stock of potential social relations 781 and improve the process of forming relationships 782. An improve stock of potential social relations 781 next level group activities 551 which, for example, could be to consider volunteering at this VA dinner 793. Solve the problem 780 improve the process of forming relationships 782 next levels examples: social skills training 571, which leads to next level examples 790 include here is a site for social skills training 791 that includes initiating conversations 574 for example here is an online listening service 792 of one embodiment.

### Network Interconnections:

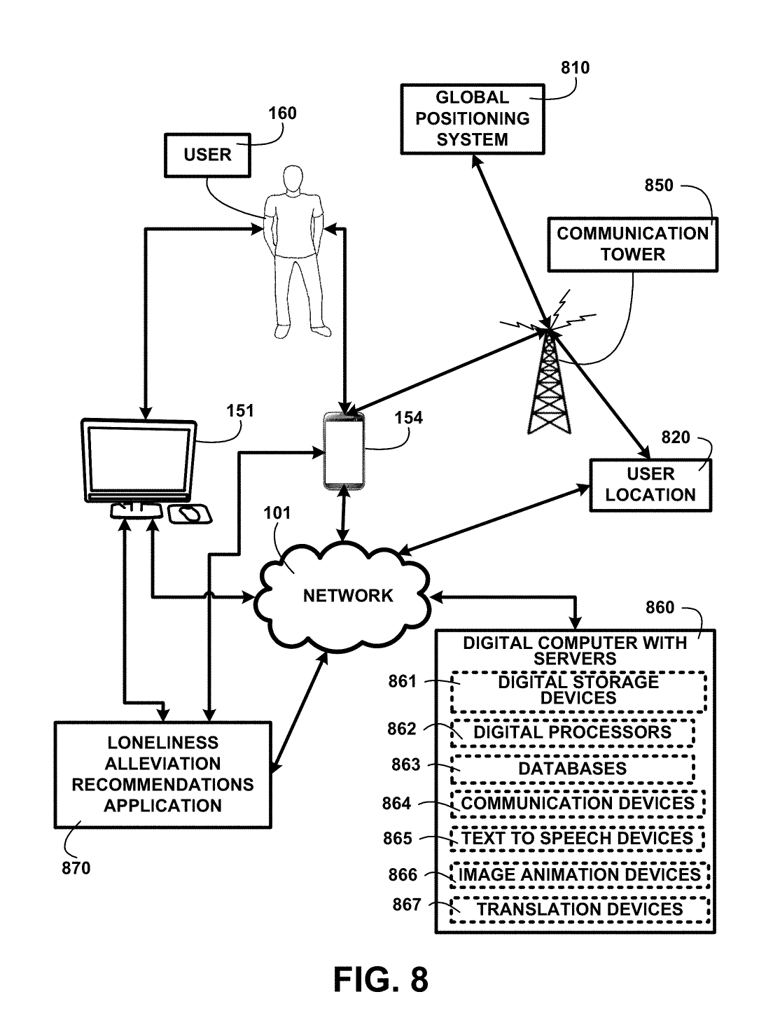

FIG. 8 shows for illustrative purposes only an example of network interconnections of one embodiment. FIG. 8 shows network interconnections from the network 101 that includes digital computer with servers 860, digital storage devices 861, and digital processors 862, databases 863 communication devices 864, text to speech devices 865, image animation devices 866, and translation devices 867. The network 101 interconnects to user devices 150 of FIG. 1 including desktop computing devices 151 and cellular phones 154 and not shown laptop computing devices 152 of FIG. 1 and tablet computing devices 153 of FIG. 1. The user devices 150 of FIG. 1 include the loneliness alleviation recommendations application 870. The user 160 receives loneliness alleviation recommendations from the network 101 through the user devices 150 of FIG. 1. The user devices 150 of FIG. 1 interconnect with the user 160 and network 101 through communications networks mounted on one or more communication tower 850 facilities and include satellite interconnections to one or more global positioning system 810 systems which provide a user location 820 of one embodiment.

### Learning Loop:

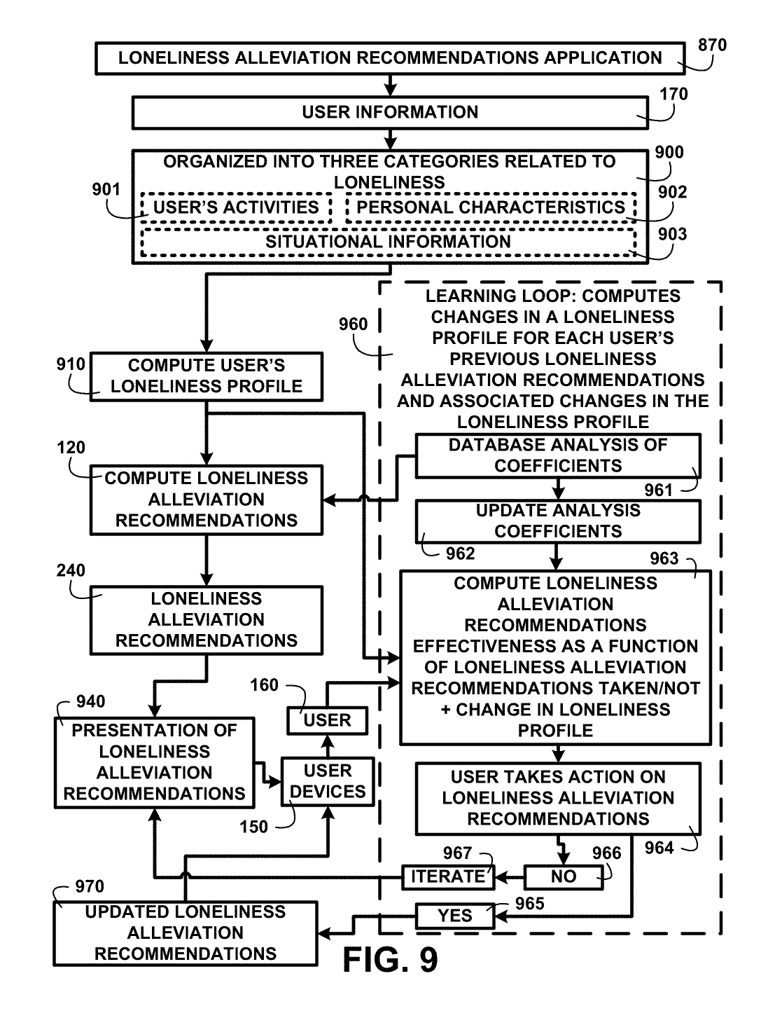

FIG. 9 shows a block diagram of an overview flow chart of learning loop of one embodiment. FIG. 9 shows the loneliness alleviation recommendations application 870 used to collect user information 170. The user information 170 is organized into three categories related to loneliness 900 including user's activities 901, personal characteristics 902, and situational information 903. The user information 170 is used to compute user's loneliness profile 910 which is then used to compute loneliness alleviation recommendations 120. The loneliness alleviation recommendations 240 are transmitted to user devices 150 for presentation of loneliness alleviation recommendations 940 to a user 160 of one embodiment.

A learning loop: computes changes in a loneliness profile for each user's previous loneliness alleviation recommendation and associated changes in the loneliness profile 960. A database analysis of coefficients 961 and an algorithm are used to update analysis coefficients 962. The network 101 of FIG. 1 is used to compute loneliness alleviation recommendations effectiveness as a function of loneliness alleviation recommendations taken or not taken plus change in loneliness profile 963 from the previous loneliness profile. If a user takes action on loneliness alleviation recommendations 964 it is recorded as a yes 965 which triggers a continuation to updated loneliness alleviation recommendations 970 then transmitted to the user device/user 950. If the user takes no action on loneliness alleviation recommendations it is recorded as a no 966 which triggers the system to iterate 967 present loneliness alleviation recommendations 940. Should the user 160 exit the application without a yes or no response then it is recorded as an exit and not updated action is taken of one embodiment

### Collect 1st Round User Information:

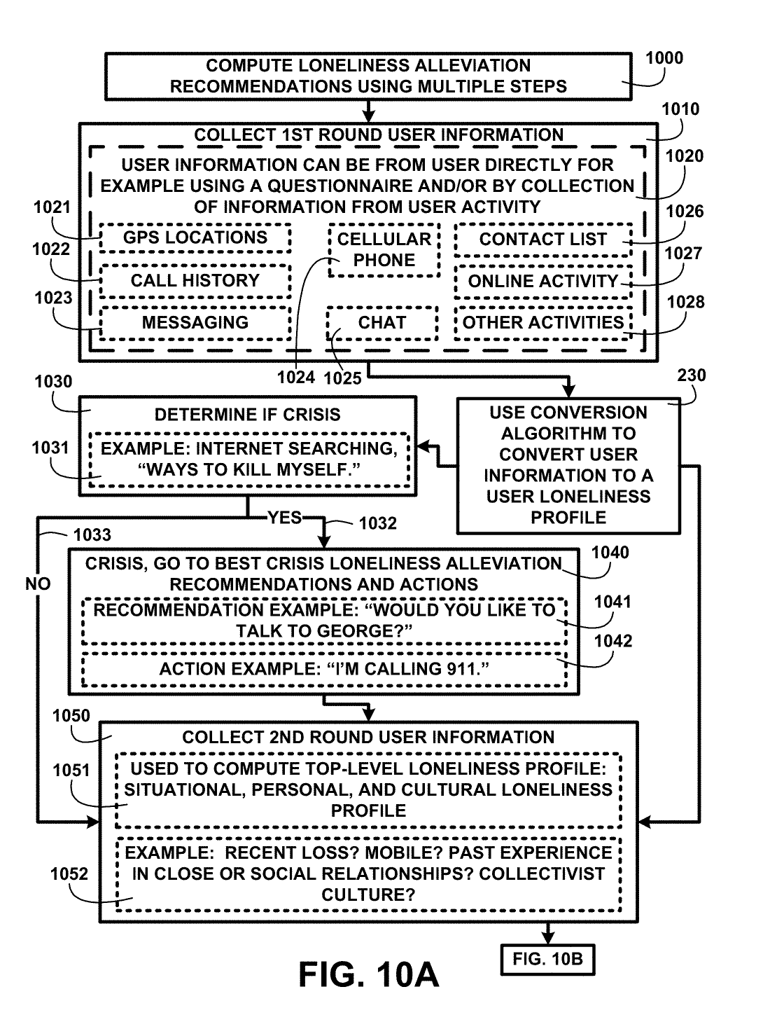

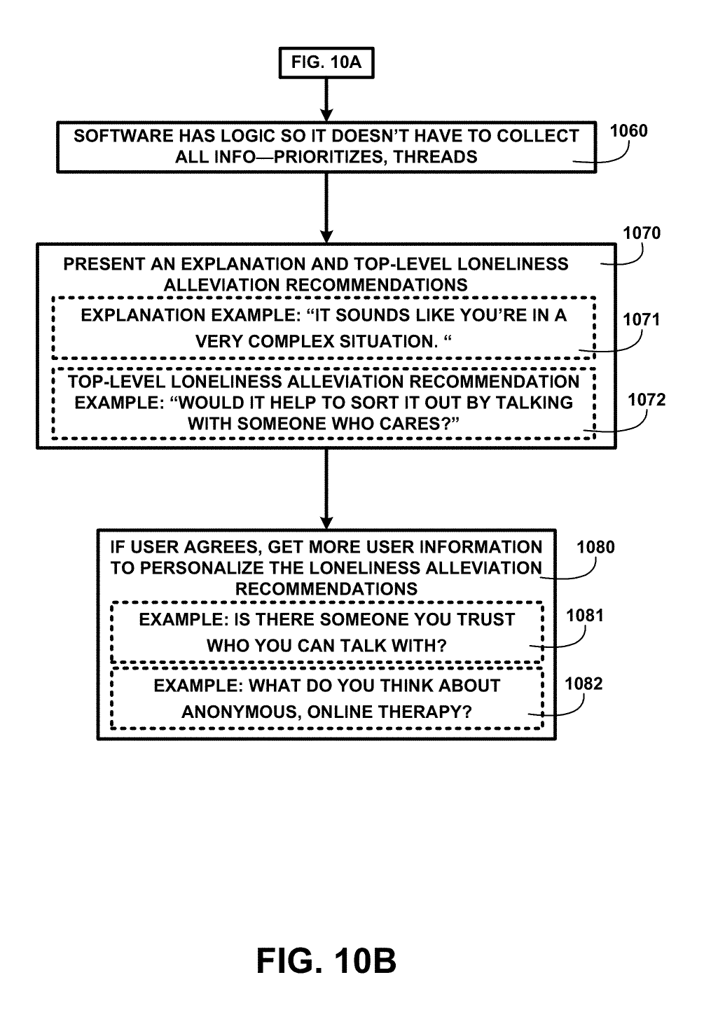

FIG. 10A shows a block diagram of an overview flow chart of collect 1st round user information of one embodiment. FIG. 10A shows a process to compute loneliness alleviation recommendations using multiple steps 1004. A first process is to collect 1st round user information 1010. User information can be from user directly for example using a questionnaire and/or by collection of information from user activity 1020. User information can also include GPS locations 1021, call history 1022, messaging 1023, cellular phone 1024, chat 1025, contact list 1026, online activity 1027, and other activities 1028. The use conversion algorithm converts user information to a user loneliness profile 230.

The first evaluation is used to determine if crisis 1030 situations exist, for example: internet searching, “ways to kill myself.” 1031. If the first evaluation determination is YES 1032 indicating the user is in a crisis, go to best crisis loneliness alleviation recommendations 1040, for a recommendation example: “would you like to talk to George?” 1041, and an action example: “I'm calling 911.” 1042.

If the first evaluation determination or a updated evaluation determination is NO 1033 indicating no crisis situation exists then the process continues to collect 2nd round user information 1050. 2nd round user information is used to compute top-level loneliness profile: situational, personal, and cultural loneliness profile 1051, for example: recent loss? mobile? past experience in close or social relationships? collectivist culture? 1052.

The first evaluation includes a crisis mode to provide quick responses. A first evaluation determination that a crisis situation exists triggers crisis loneliness alleviation recommendations and actions. Thus, the system is configured to ONLY take action if a crisis exists creating a differentiation between crisis mode and non-crisis mode. The crisis mode is important since time is of the essence in any crisis. In one embodiment parents can install the loneliness alleviation recommendations application 870 of FIG. 8 on their depressed children's user devices for example a cellular phone 154 of FIG. 1. In this embodiment the parents can activate a link from the child's user device to their own user devices 150 of FIG. 1 to directly report to the parents any first evaluation determination indicating a crisis mode exists to provide a quick response loneliness alleviation recommendation early warning to seek medical help of one embodiment. The process description continues on FIG. 10B,

### Top-level Loneliness Alleviation Recommendations:

FIG. 10B shows a block diagram of an overview flow chart of top-level loneliness alleviation recommendations of one embodiment. FIG. 10B shows a continuation from FIG. 10A showing that the software has logic so it doesn't have to collect all info—prioritizes, threads 1060. The network 101 of FIG. 1 transmits the data to present an explanation and top-level loneliness alleviation recommendations 1070, an explanation example: “it sounds like you're in a very complex situation.” 1071. A top-level loneliness alleviation recommendation example: “would it help to sort it out by talking with someone who cares?” 1072. If user agrees, get more user information to personalize the loneliness alleviation recommendations 1080, for example: is there someone you trust who you can talk with? 1081 and for example: what do you think about anonymous, online therapy? 1082 of one embodiment.

### Data Analysis Method:

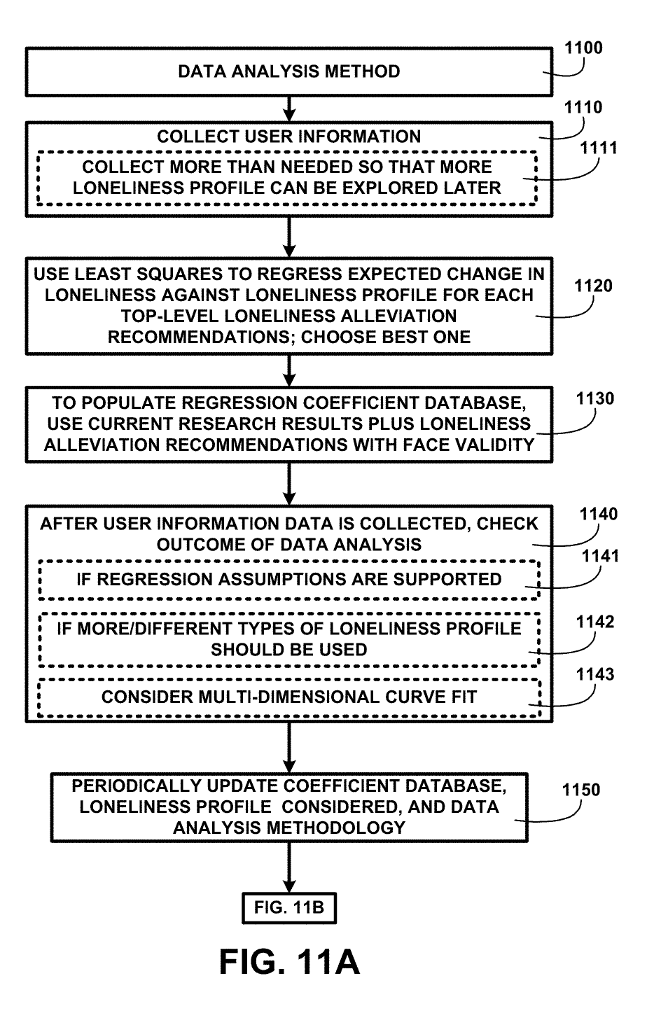

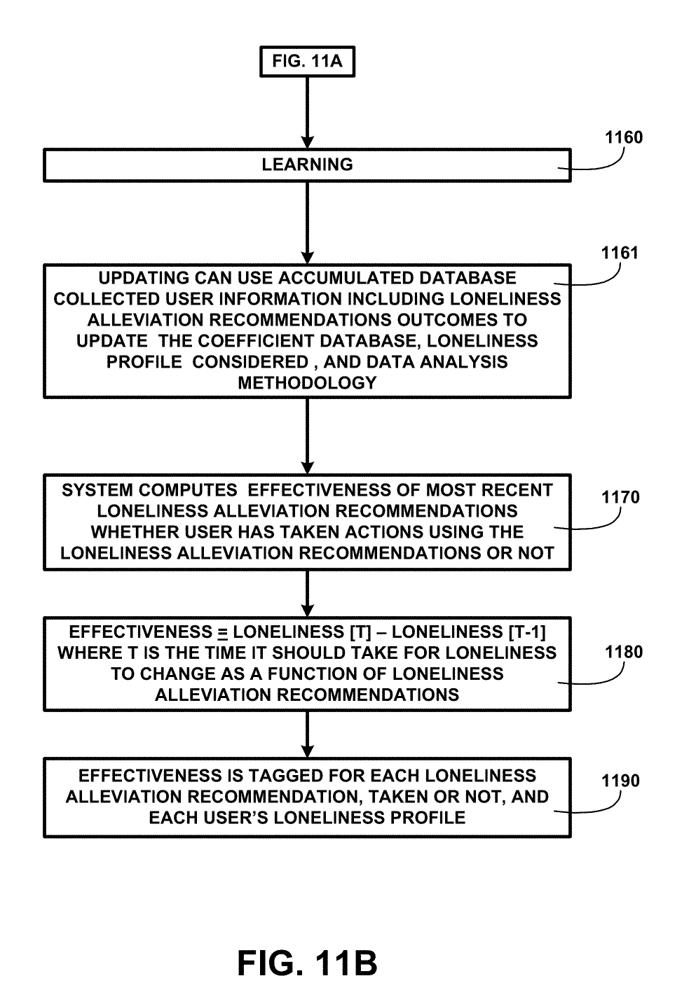

FIG. 11A shows a block diagram of an overview flow chart of data analysis method of one embodiment. FIG. 11A shows a data analysis method 1100 starting with a process to collect user information 1110. The process is used to collect more than needed so that more loneliness profile can be explored later 1111. The data analysis method 1100 can use least squares to regress expected change in loneliness against loneliness profile for each top-level loneliness alleviation recommendations; choose best one 1120. The data analysis method 1100 can be used to initially populate regression coefficient database, use current research results plus loneliness alleviation recommendations with face validity 1130. After user information data is collected, data mining techniques can be used for check outcome of data analysis 1140 including if regression assumptions are supported 1141, if more/different types of loneliness profile should be used 1142, and consider multi-dimensional curve fit 1143. The process can be repeated to periodically update coefficient database, loneliness profile considered, and data analysis methodology 1150 of one embodiment. The process description is continued on FIG. 11B.

### Learning:

FIG. 11B shows a block diagram of an overview flow chart of learning of one embodiment. FIG. 11B shows a continuation from FIG. 11A to show process learning 1160. Updating can use accumulated database collected user information including loneliness alleviation recommendations outcomes to update the coefficient database, loneliness profile considered, and data analysis methodology 1161. A process system computes effectiveness of most recent loneliness alleviation recommendations whether user has taken actions using the loneliness alleviation recommendations or not 1170. Effectiveness=loneliness [t]−loneliness [t−1] where t is the time it should take for loneliness to change as a function of loneliness alleviation recommendations 1180. Effectiveness is tagged for each loneliness alleviation recommendation, taken or not, and each user's loneliness profile 1190 of one embodiment.

### Virtual Buddy and User Interaction:

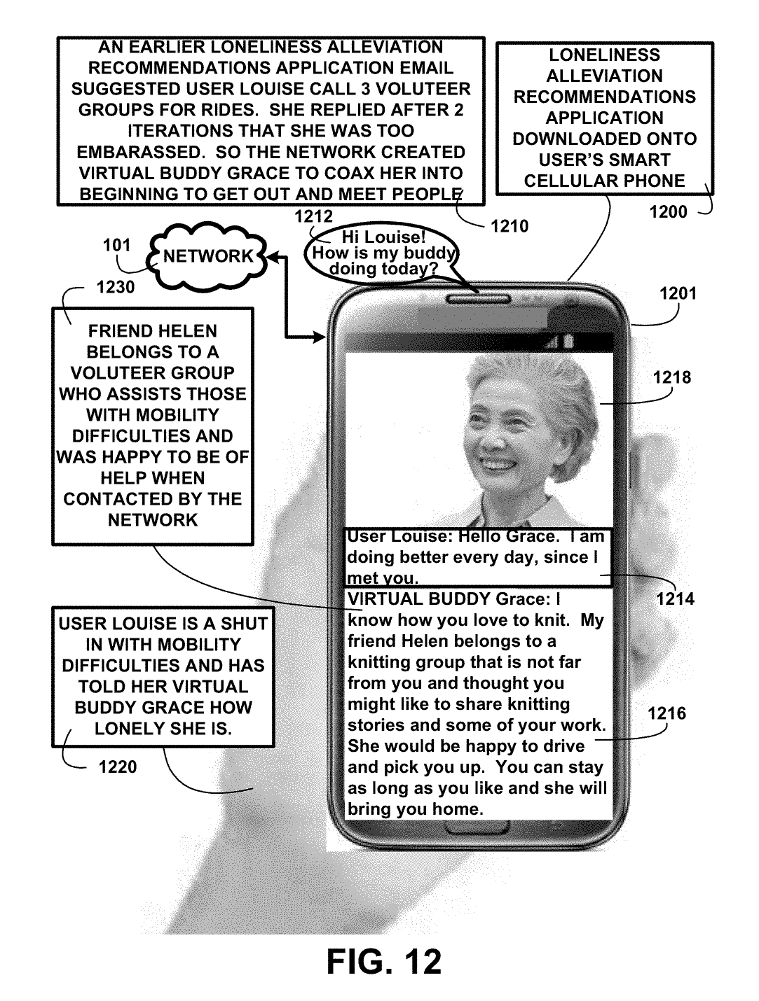

FIG. 12 shows for illustrative purposes only an example of Virtual Buddy and user interaction of one embodiment. FIG. 12 shows a loneliness alleviation recommendations application downloaded onto user's smart cellular phone 1200 and connected to the network 101.

An earlier loneliness alleviation recommendations application email suggested user Louise calls 3 volunteer groups for rides. She replied after 2 iterations that she was too embarrassed. So the network created Virtual Buddy Grace to coax her into beginning to get out and meet people 1210. User Louise answers a call from Virtual Buddy Grace on her smart phone 1201. The screen shows a Virtual Buddy Grace animated image 1218, and vocalizes

“Hi Louise! How is my buddy doing today?” 1212. User Louise: “Hello grace. I am doing better every day, since I met you.” 1214. Virtual Buddy Grace: “I know how you love to knit. My friend Helen belongs to a knitting group that is not far from you and thought you might like to share knitting stories and some of your work. She would be happy to drive and pick you up. You can stay as long as you like and she will bring you home.” 1216. User Louise is a shut-in with mobility difficulties and has told her Virtual Buddy Grace how lonely she is 1220. Friend Helen belongs to a volunteer group who assists those with mobility difficulties and was happy to be of help when contacted by the network 1230 of one embodiment.

### Web Resource Social Skills Training:

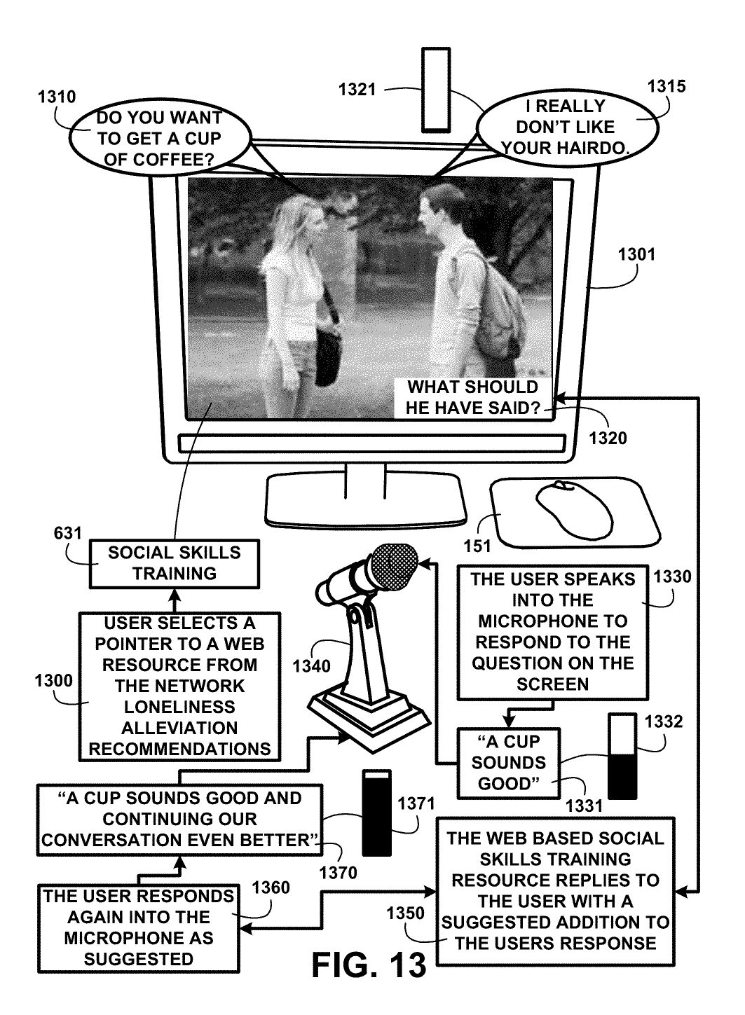

FIG. 13 shows for illustrative purposes only an example of web resource social skills training of one embodiment. FIG. 13 shows where a user selects a pointer to a web resource from the network loneliness alleviation recommendations 1300 for social skills training 601. FIG. 13 shows web resource social skills training playing on user device with the loneliness alleviation recommendations application installed 1301. The user device is one of the desktop computing devices 151. The web resource social skills training plays a scene in which a woman asks a man “Do you want to get a cup of coffee?” 1310, to which the man replies “I really don't like your hairdo.” 1315. The social skills training shows a response quality meter near zero 1321. The social skills training displays a question to the user “what should he have said?” 1320. The user speaks into the microphone to respond to the question on the screen 1330 and responds “a cup sounds good” 1331. A microphone 1340 is plugged into the desktop computer. The social skills training displays a response quality meter at mid-point value 1332. The web based social skills training resource replies to the user with a suggested addition to the user's response 1350. The user responds again into the microphone as suggested 1360, “a cup sounds good and continuing our conversation even better” 1370, a response quality meter near top value 1371 is displaying indicating the user progressed in his response of one embodiment.

### Loneliness Alleviation Recommendations Application:

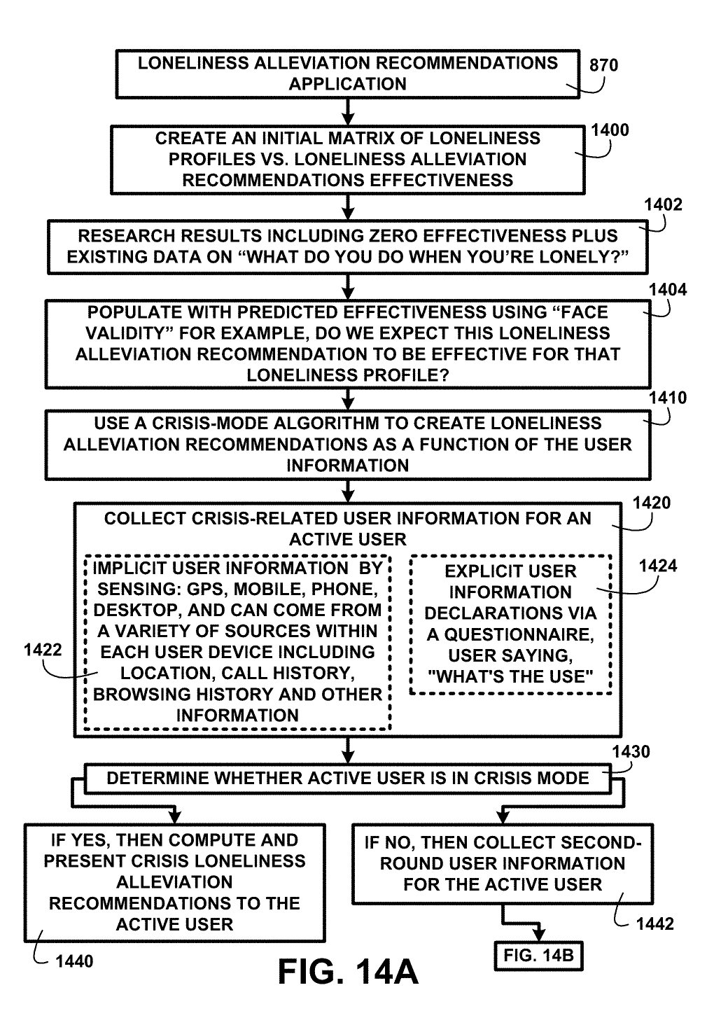

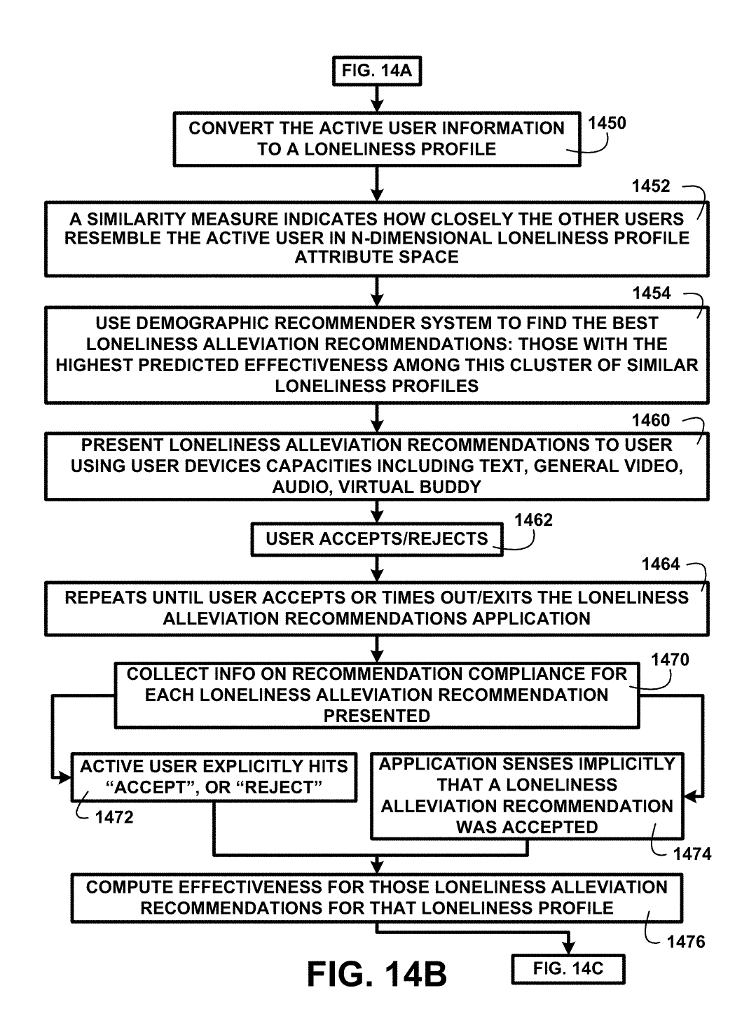

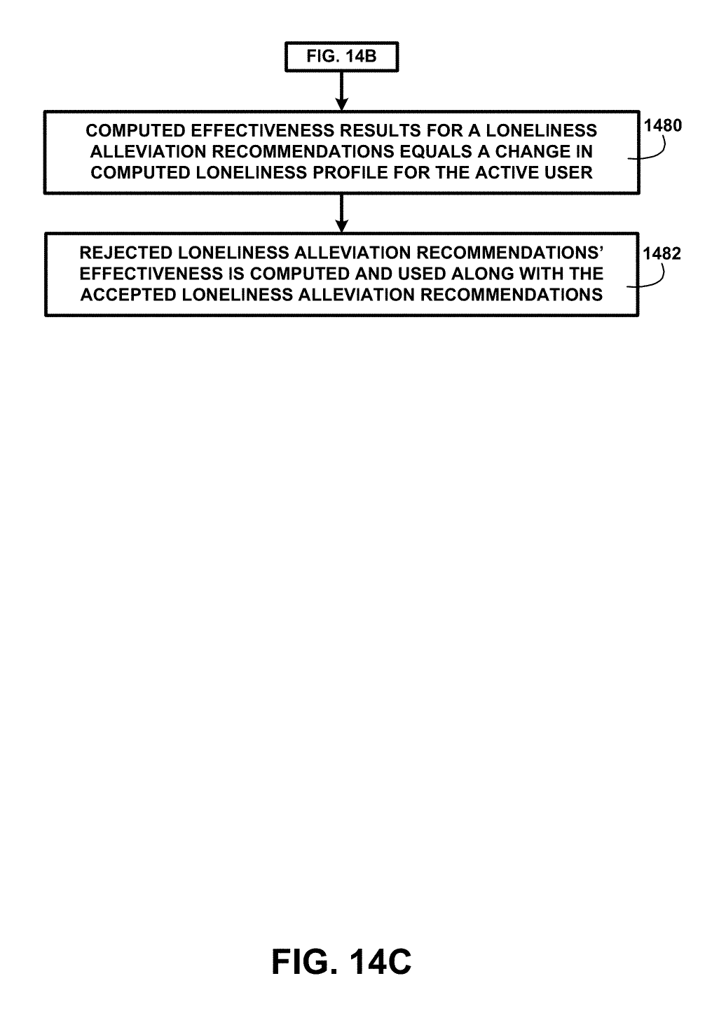

FIG. 14A shows a block diagram of an overview flow chart of loneliness alleviation recommendations application of one embodiment. FIG. 14A shows the loneliness alleviation recommendations application 870. The loneliness alleviation recommendations application 870 is used to create an initial matrix of loneliness profiles vs, loneliness alleviation recommendations effectiveness 1400 using research results including zero effectiveness plus existing data on “what do you do when you're lonely?” 1402 to populate with predicted effectiveness using “face validity” for example; do we expect this loneliness alleviation recommendation to be effective for that loneliness profile? 1404.

The system and method of generating recommendations to alleviate loneliness can use a crisis-mode algorithm to create loneliness alleviation recommendations and actions as a function of the user information 1410 and collect crisis-related user information for an active user 1420. User information collected can include implicit user information by sensing: GPS, mobile, phone, desktop, and can come from a variety of sources within each user device including location, call history, browsing history and other information 1422 and explicit user information declarations via a questionnaire, user saying, “What's the use” 1424. The first process is to determine whether active user is in crisis mode 1430, if yes, then compute and present crisis loneliness alleviation recommendations or take crisis actions to the active user 1440. If no, then collect second-round user information for the active user 1442 of one embodiment. The description continues on FIG. 14B.

### Convert the Active User Information to a Loneliness Profile:

FIG. 14B shows a block diagram of an overview flow chart of convert the active user information to a loneliness profile of one embodiment. FIG. 14B shows the description continuing from FIG. 14A including the process to convert the active user information to a loneliness profile 1450. The process can use active user's loneliness profile to find a cluster of similar loneliness profiles for example those of past users whose loneliness profiles are similar to active user's to create a similarity measure which indicates how closely the other users resemble the active user in n-dimensional loneliness profile attribute space 1452. The process can use a demographic recommender system to find the best loneliness alleviation recommendations: those with the highest predicted effectiveness among this cluster of similar loneliness profiles 1454. The process can use various formats to present loneliness alleviation recommendations to user using user devices' capacities including text, general video, audio, Virtual Buddy 1460. A user accepts/rejects 1462 the loneliness alleviation recommendations presented. The process repeats until user accepts or times out/exits the loneliness alleviation recommendations application 1464. The process can collect info on recommendation compliance for each loneliness alleviation recommendation presented 1470 including an active user explicitly hits “accept”, or “reject” 1472. The application can sense implicitly that a loneliness alleviation recommendation was accepted 1474 and can compute effectiveness for those loneliness alleviation recommendations for that loneliness profile 1476 of one embodiment. The description continues on FIG. 14C.

### Computed Effectiveness Results:

FIG. 14C shows a block diagram of an overview flow chart of computed effectiveness results of one embodiment. FIG. 14C shows a continuation from FIG. 14B where computed effectiveness results for loneliness alleviation recommendations equals a change in computed loneliness profile for the active user 1480. Rejected loneliness alleviation recommendations' effectiveness is computed and used along with the accepted loneliness alleviation recommendations 1482.

This effectiveness information is used as follows: a new, active “user” is created in the matrix of loneliness profile vs effectiveness. The data for this user consists of the user's initial loneliness profile and the effectiveness of that recommendation. Additionally the effectiveness information may be used to update the algorithm and databases for converting user information to loneliness profiles, or the algorithm and coefficient database for the structural equation algorithm or the algorithm and database for the recommender system calculation. For example, the structural algorithm could use regression analysis. When additional effectiveness data is obtained and mined, the number of predictor variables or nonlinearity degrees can be updated. Finally, new predictor methodology based on data-mining techniques may be determined to be more useful.

Loneliness profiles including changes are indexed by loneliness alleviation recommendations computed effectiveness results 1484. The conversion from user information to loneliness profiles uses functions including a weights database, structural equations, or recommender system database and algorithms. Data mining techniques are used to obtain updates for these databases, equations, and algorithms of one embodiment.

### Algorithm Functions:

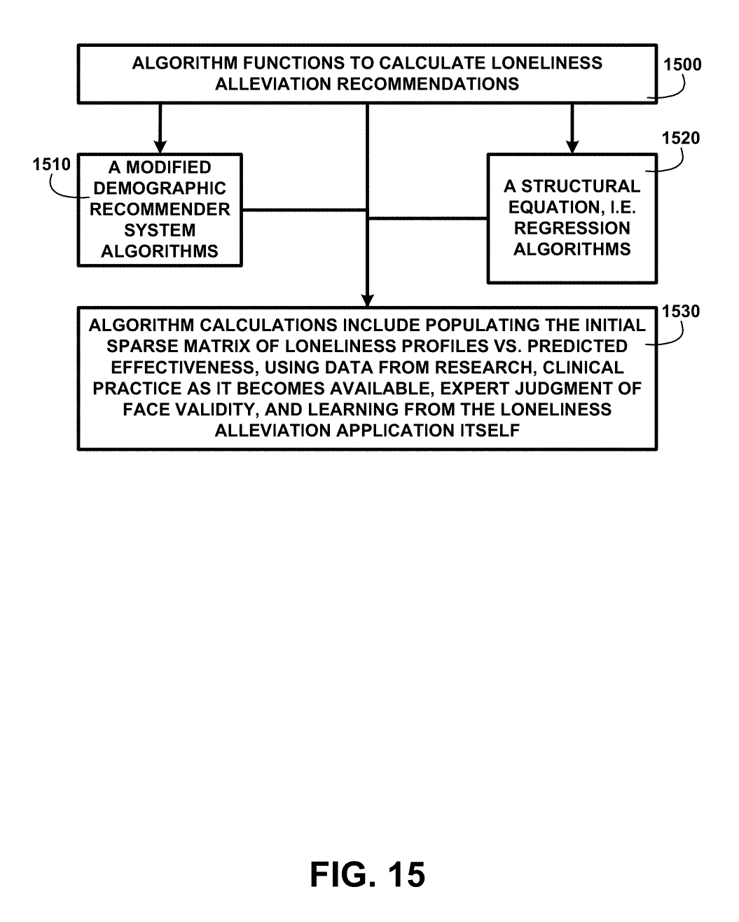

FIG. 15 shows a block diagram of an overview an example of algorithm functions of one embodiment. FIG. 15 shows algorithm functions to calculate loneliness alleviation recommendations 1500. Algorithms can include a modified demographic recommender system algorithms 1510 or a structural equation, i.e. regression algorithms 1520. Algorithm calculations 1510 and 1520 include populating the initial sparse matrix of loneliness profiles vs. predicted effectiveness, using data from research, clinical practice as it becomes available, expert judgment of face validity, and learning from the loneliness alleviation application itself 1530 of one embodiment.

### Other Implicit User Information Devices:

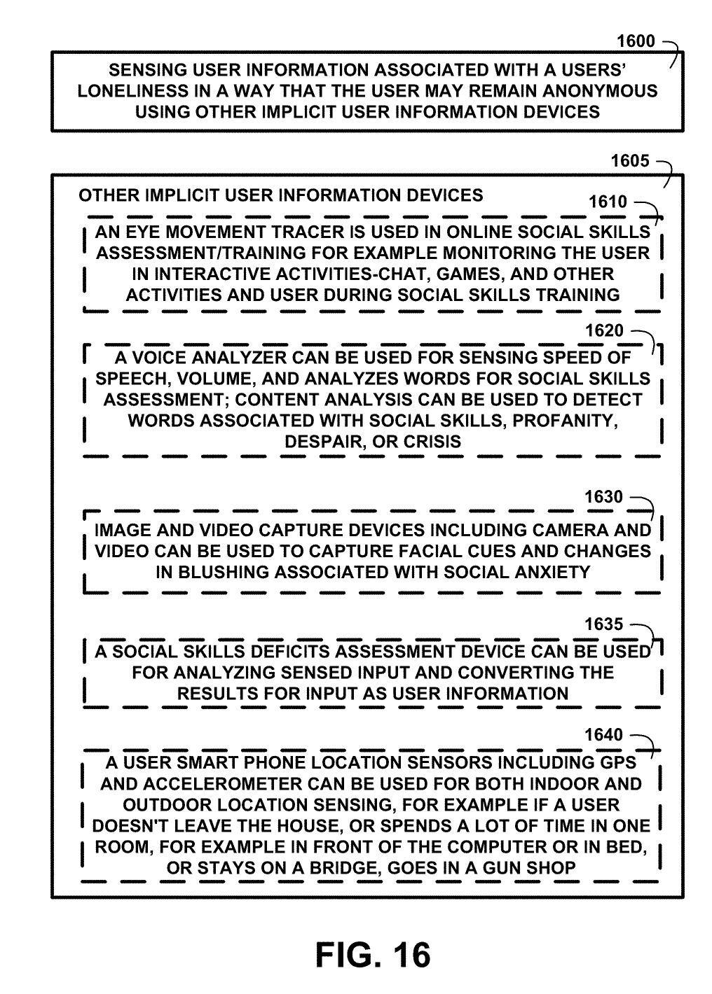

FIG. 16 shows a block diagram of an overview an example of other implicit user information devices of one embodiment. FIG. 16 shows sensing user information associated with a users' loneliness in a way that the user may remain anonymous using other implicit user information devices 1600. Other implicit user information devices 1605 include the following: An eye movement tracer is used in online social skills assessment/training for example monitoring the user in interactive activities-chat, games, and other activities and user during social skills training 1610;

A voice analyzer can be used for sensing speed of speech, or volume, and analyzes words for social skills assessment; Content analysis can be used to detect words associated with social skills, profanity, despair, or crisis 1620; Image and video capture devices including camera and video can be used to capture facial cues and changes in blushing associated with social anxiety 1630; A social skills deficits assessment device can be used for analyzing sensed input and converting the results for input as user information 1635; and a user smart phone location sensors including GPS and accelerometer can be used for both indoor and outdoor location sensing, for example if a user doesn't leave the house, or spends a lot of time in one room, for example in front of the computer or in bed, or stays on a bridge, goes in a gun shop 1640 of one embodiment.

### Modified Demographic Recommender System Data Analysis Method:

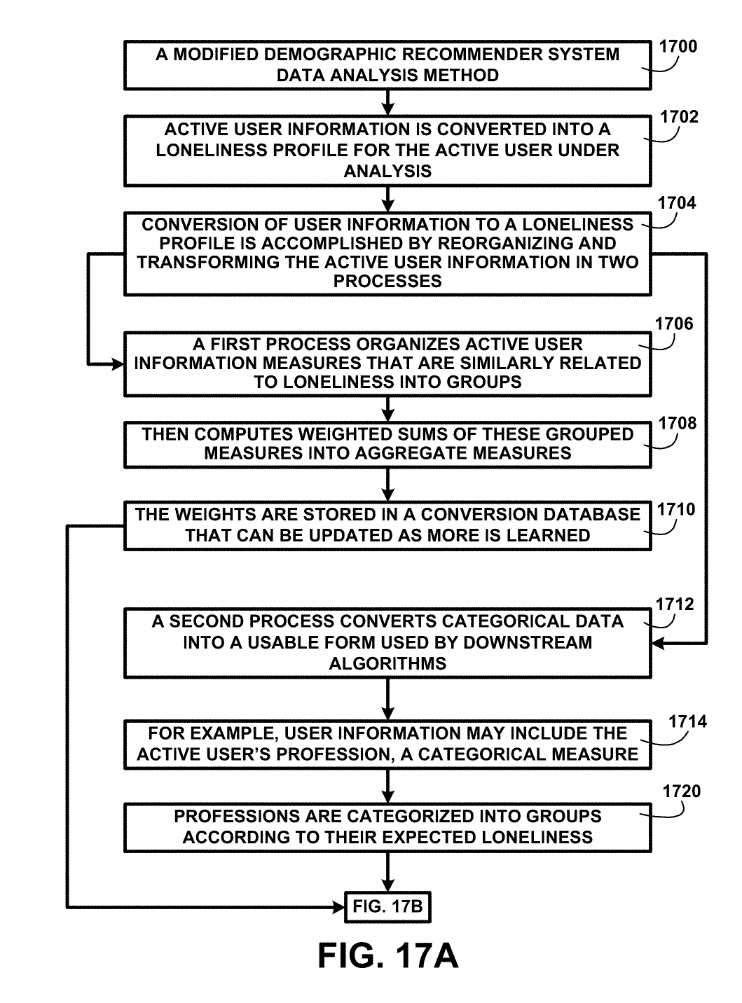

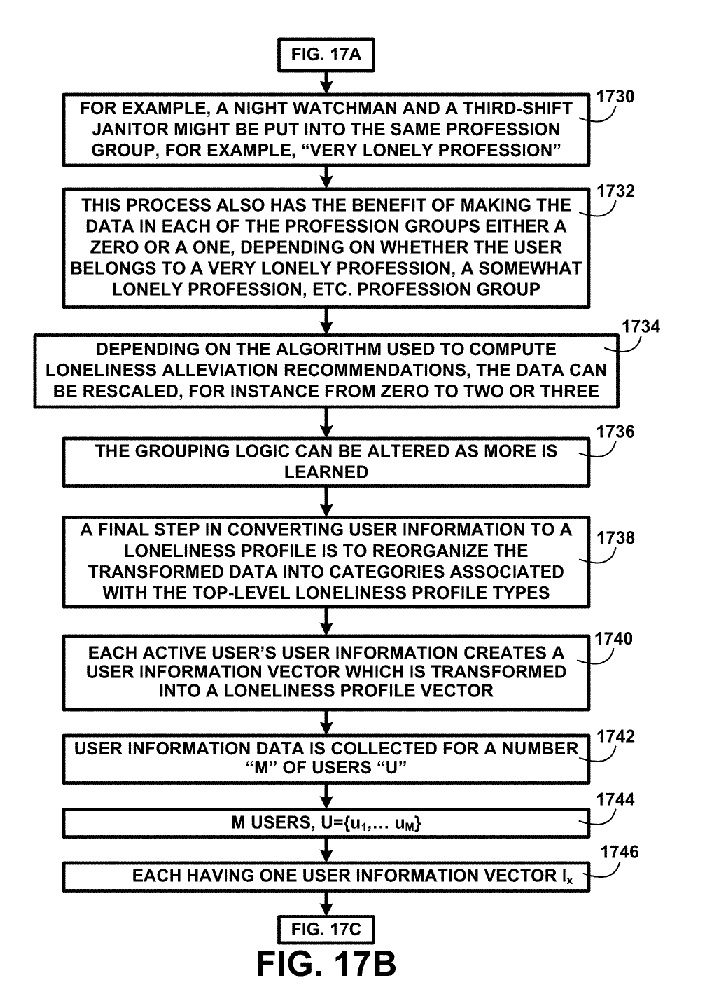

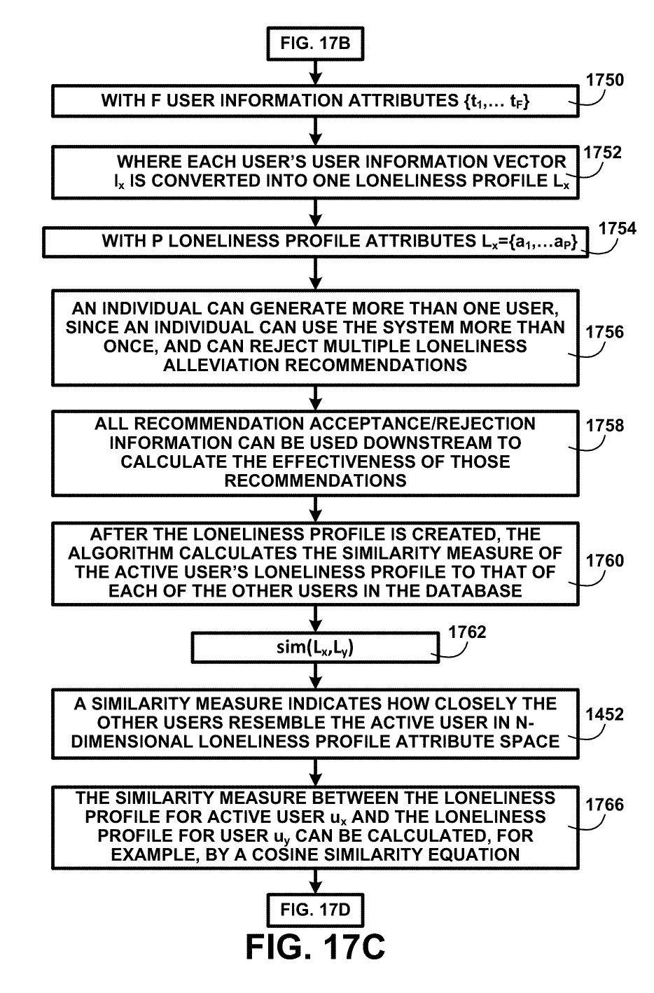

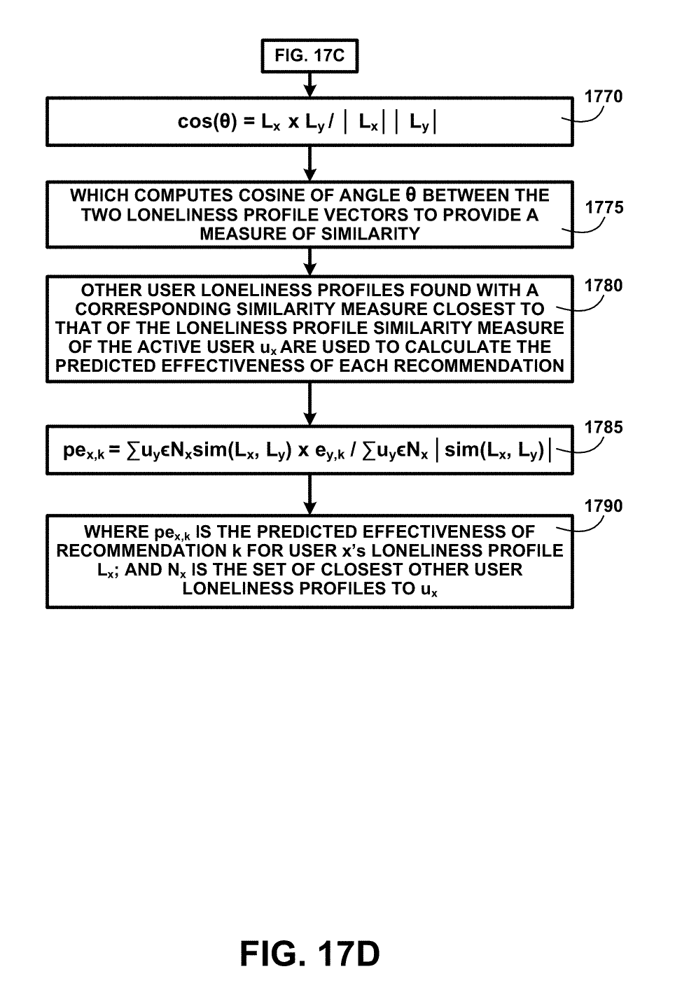

FIG. 17A shows a block diagram of an overview flow chart of a modified demographic recommender system data analysis method of one embodiment. FIG. 17A shows a modified demographic recommender system data analysis method 1700. Active user information is converted into a loneliness profile for the active user under analysis 1702. Conversion of user information to a loneliness profile is accomplished by reorganizing and transforming the active user information in two processes 1704. A first process organizes active user information measures that are similarly related to loneliness into groups 1706, then computes weighted sums of these grouped measures into aggregate measures 1708. The weights are stored in a conversion database that can be updated as more is learned 1710.

A second process converts categorical data into a usable form used by downstream algorithms 1712. For example, user information may include the active user's profession, a categorical measure 1714. Professions are categorized into groups according to their expected loneliness 1720 of one embodiment. The description continues on FIG. 17B.

### User Professions Expected Loneliness Groups Categorization Analysis Process:

FIG. 17B shows a block diagram of an overview flow chart of a user professions expected loneliness groups categorization analysis process of one embodiment. FIG. 17B shows a continuation from FIG. 17A showing for example, a night watchman and a third-shift janitor might be put into the same profession group, for example, “very lonely profession” 1730. This process also has the benefit of making the data in each of the profession groups either a zero or a one, depending on whether the user belongs to a very lonely profession, a somewhat lonely profession, etc. profession group 1732. Depending on the algorithm used to compute loneliness alleviation recommendations, the data can be rescaled, for instance from zero to two or three 1734. The grouping logic can be altered as more is learned 1736.

A final step in converting user information to a loneliness profile is to reorganize the transformed data into categories associated with the top-level loneliness profile types 1738. Each active user's user information creates a user information vector which is transformed into a loneliness profile vector 1740. An overall loneliness score is computed by taking the Loneliness Profile vector and multiplying it by yet another vector of weights. User information data is collected for a number “M” of users “U” 1742, with M users, U={u₁, . . . u_{M}} 1744 each having one user information vector Iₓ 1746 of one embodiment. The processing description continues on FIG. 17C.

### User Information to Loneliness Profile Conversion Process:

FIG. 17C shows a block diagram of an overview flow chart of a user information to loneliness profile conversion process of one embodiment. FIG. 17C shows a continuation from FIG. 17B with F user information attributes {t₁, . . . t_{f}}1750, where each user's user information vector Iₓ is converted into one loneliness profile Lₓ 1752 with P loneliness profile attributes Lₓ={a₁, . . . a_{P}} 1754. An individual can generate more than one user, since an individual can use the system more than once, and can reject multiple loneliness alleviation recommendations 1756. All recommendation acceptance/rejection information can be used downstream to calculate the effectiveness of those recommendations 1758. After the loneliness profile is created, the algorithm calculates the similarity measure of the active user's loneliness profile to that of each of the other users in the database 1760, sim (Lx, Ly) 1762. A similarity measure indicates how closely the other users resemble the active user in N-dimensional loneliness profile attribute space 1452. The similarity measure between the loneliness profile for active user uₓ, and the loneliness profile for user u_{y} can be calculated, for example, by a cosine similarity equation 1766 of one embodiment. The description of the processing continues on FIG. 17D.

### Loneliness Profile Vectors Measure of Similarity Analysis Process:

FIG. 17D shows a block diagram of an overview flow chart of a loneliness profile vectors measure of similarity analysis process of one embodiment. FIG. 17D shows continuing from FIG. 17C the cosine similarity equation cos(θ)=Lx·Ly/|Lx| |Ly| 1770, which computes cosine of angle θ between the two loneliness profile vectors to provide a measure of similarity 1775. Other user loneliness profiles found with a corresponding similarity measure closest to that of the loneliness profile similarity measure of the active user uₓ are used to calculate the predicted effectiveness of each recommendation 1780. The predicted effectiveness of each recommendation is calculated using 1785, where pe_{x,k} is the predicted effectiveness of pe_{x,k}=Σu_{y}∈Nₓsim(Lₓ,L_{y})×e_{y,k}/Σu_{y}∈Nₓ|sim(Lₓ,L_{y})| recommendation k for user x's loneliness profile Lₓ; and Nₓ is the set of closest other user loneliness profiles to uₓ 1790 of one embodiment.

The foregoing has described the principles, embodiments and modes of operation of the embodiments, However, the embodiments should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. A system and method of generating recommendations to alleviate loneliness comprising:
detecting, sensing, collecting, and organizing user information related to loneliness including computing a user's loneliness profiles associated with a users' loneliness in a way that the user may remain anonymous, and a loneliness alleviation recommendations application for communicating between a loneliness alleviation network and a user using at least one user device with the loneliness alleviation recommendations application installed;
converting the user information into a loneliness profile;
processing a loneliness profile crisis mode first evaluation for determining if a crisis situation exists and if it exists then for providing quick responses including loneliness alleviation recommendations and actions;
creating a modified demographic recommender system data analysis method including a first process for organizing active user information measures that are similarly related to loneliness into groups and a second process for converting categorical data into a usable form used by downstream algorithms;
using the user loneliness profile to compute one or more loneliness alleviation recommendations;
presenting the one or more loneliness alleviation recommendations to the user;
analyzing the effectiveness of the one or more loneliness alleviation recommendations;
using effectiveness analysis results and user loneliness profiles to compute and store updates to databases and algorithms that compute loneliness alleviation recommendations;
converting a user's updated loneliness profile from updated user information and one or more loneliness alleviation recommendations effectiveness analysis results; and;
using the updated user information and updated user loneliness profile to compute one or more updated loneliness alleviation recommendations.

2. The method of claim 1, wherein user loneliness profiles are converted using user information data from the user's online activities and the user's offline activities.

3. The method of claim 1, wherein user information is used to create a user information vector which is transformed into a loneliness profile vector and wherein an overall loneliness score is computed by taking a loneliness profile vector and multiplying it by a vector of weights.

4. The method of claim 1, wherein the one or more loneliness alleviation recommendations to alleviate user's loneliness are computed using user loneliness profiles.

5. The method of claim 1, wherein the effectiveness of the one or more loneliness alleviation recommendations is computed by evaluating any changes in a user's loneliness profile.

6. The method claim 1, wherein the one or more loneliness alleviation recommendations is computed by including information about what loneliness alleviation recommendations have not been effective previously.

7. The method of claim 1 wherein new information from research, clinical practice, and the system and method of generating recommendations to alleviate loneliness effectiveness analysis is used to update the network databases that are used to compute user loneliness profiles and loneliness alleviation recommendations.

8. The method of claim 1, wherein the effectiveness of the one or more loneliness alleviation recommendations are used to update network databases and adjust algorithms including changes in underlying structural equations or recommender system databases that are used to compute loneliness alleviation recommendations.

9. The method of claim 1, wherein the one or more loneliness alleviation recommendations include social skills training including network-delivered online social skills training using the loneliness alleviation recommendations application on a user device.

10. The method of claim 1, wherein sensing loneliness profiles associated with a users' loneliness in a way that the user may remain anonymous includes using at least one other implicit user information devices including an eye movement tracer, voice analysis, image and video capture including a camera and video recorder, and a user smart cellular phone location sensors including GPS and accelerometer.

11. An apparatus, comprising:
a loneliness alleviation network for detecting, sensing, collecting, and organizing user information related to loneliness, converting the user information into one or more user loneliness profile using an algorithm, computing and presenting to a user at least one loneliness alleviation recommendation, analyzing user actions and responses to the at least one loneliness alleviation recommendation,
wherein the loneliness alleviation network includes a digital computer with servers, digital storage devices, digital processors, algorithms, databases, communication devices, text to speech devices, image animation devices, translation devices, other implicit user information devices including an eye movement tracer, voice analysis, image and video capture including a camera and video recorder, and a user smart cellular phone location sensors including GPS and accelerometer, at least one user device with a loneliness alleviation recommendations application installed on a user device;
wherein the digital processors and algorithms are used for analyzing the at least one loneliness alleviation recommendations effectiveness;
wherein communication devices include the capacity for presenting to a user at least one loneliness alleviation recommendation; and;
wherein the at least one user device includes the capacity to receive one or more loneliness alleviation recommendations presentation in text and other formats including video, audio and Virtual Buddy virtual character formats and to transmit to the network in text and other formats including video, and audio a user response to the one or more loneliness alleviation recommendations.

12. The apparatus of claim 11, wherein algorithms are configured to include functions to compute loneliness alleviation recommendations from user's loneliness profiles using demographic recommender system algorithms and in an alternative embodiment structural equation algorithms, and algorithm calculations to populate the initial sparse matrix of loneliness profiles vs. predicted effectiveness, using data from research, clinical practice as it becomes available, expert judgment of face validity, and learning from the loneliness alleviation application itself and are configured to include converting the user information into a loneliness profile.

13. The apparatus of claim 11, wherein the at least one user device includes at least one desktop computing devices, laptop computing devices, tablet computing devices, and cellular phones including smart cellular phones.

14. The apparatus of claim 11, wherein the network including the text to speech devices, image animation devices, translation devices are configured to create a virtual friend used as a user-assigned Virtual Buddy including a name, animated image, voice and a computer generated persona, wherein the Virtual Buddy can be programmed to communicate with user via emails and texts, and alternatively the Virtual Buddy can be programmed to call, video chat daily or more frequently with user using a virtual voice.

15. The apparatus of claim 11, wherein the network analyzing a change in a user loneliness profile using the at least one loneliness alleviation recommendations user responses is configured for updating the computing of next updated loneliness alleviation recommendations.

16. An apparatus, comprising:
at least one device for presenting to a user at least one loneliness alleviation recommendation to assist the user in overcoming the user's sense of loneliness;
at least one user device for receiving presentations of at least one loneliness alleviation recommendation and responding to the at least one loneliness alleviation recommendation and transmitting to a network user information;
a network including at least one digital computer with servers, digital storage devices, digital processors, algorithms, databases, communication devices, text to speech devices, image animation devices, translation devices, other implicit user information device including an eye movement tracer, voice analysis, image and video capture including a camera and video recorder configured for detecting, collecting, and organizing user information related to loneliness, computing one or more user loneliness profile, computing and presenting to a user at least one loneliness alleviation recommendation, analyzing user actions and responses to the at least one loneliness alleviation recommendation, updating updated user loneliness profiles and loneliness alleviation recommendations using the analysis; and;
wherein the presentations to a user are configured to include text, emails, video, audio and a network created virtual friend format including a user specified Virtual Buddy.

17. The apparatus of claim 16, wherein network devices used for analyzing a user loneliness profile and responses to the at least one loneliness alleviation recommendations are configured for updating any changes in the computing of updated user loneliness profiles and computing updated loneliness alleviation recommendations using the computed updated user loneliness profiles.

18. The apparatus of claim 16, wherein network devices are configured for creating a virtual character used as a user-assigned Virtual Buddy including a name, animated image, voice and a computer generated persona, wherein the Virtual Buddy can be programmed to communicate with user frequently via emails and texts, the Virtual Buddy can be programmed to call, video chat daily or more frequently with user using a virtual voice.

19. The apparatus of claim 16, wherein algorithms are configured to include functions to compute loneliness alleviation recommendations from user's loneliness profiles using demographic recommender system algorithms and in an alternative structural equation algorithms, and algorithm calculations to populate the initial sparse matrix of loneliness profiles vs. predicted effectiveness, using data from research, clinical practice as it becomes available, expert judgment of face validity, and learning from the loneliness alleviation application itself and are configured to include converting the user information into a loneliness profile.

20. The apparatus of claim 16, wherein network devices are configured for gathering effectiveness data to use in updating weighting function databases, coefficient databases, structural equation characteristics, types of user information to collect, types of loneliness profile attributes.
